(19) United States
(12) Patent Application Publication
(10) Pub. No.: US 2026/0098265 A1
(43) Pub. Date: Apr. 9, 2026

(54) COMPOSITIONS AND METHODS FOR TREATMENT OF SPINOCEREBELLAR ATAXIAS

(71) Applicant: Atalanta Therapeutics, Inc., Boston, MA (US)

(72) Inventors: Corrie GALLANT-BEHM, Wellesley, MA (US); Wimalanathan KOKULAPALAN, Cambridge, MA (US); Matthew HASSLER, Boston, MA (US); Daniel CURTIS, Belmont, MA (US); Bruno Miguel DA CRUZ GODINHO, Reading, MA (US)

(21) Appl. No.: 19/115,439

(22) PCT Filed: Sep. 28, 2023
(86) PCT No.: PCT/US2023/075414

§ 371 Date: Mar. 26, 2025

Related U.S. Application Data
(60) Provisional application No. 63/377,469, filed on Sep. 28, 2022.

(51) Int. Cl. C12N 15/113

(52) U.S. Cl. CPC C12N 15/113; C12N 2310/14; C12N 2310/52

(57) ABSTRACT

The present disclosure provides single- or double-stranded interfering RNA molecules (e.g., siRNA) that target an ataxin-3 (ATXN3) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a spinocerebellar ataxia (e.g., spinocerebellar ataxia type 3).

### SEQUENCE LISTING

The instant application contains a Sequence Listing which has been submitted electronically in XML format and is hereby incorporated by reference in its entirety. Said XML copy, created on Sep. 19, 2023, is named “51436-038WO2_Sequence_Listing_9_19_23” and is 505,856 bytes in size.

### TECHNICAL FIELD

This disclosure relates to small interfering RNA (siRNA) molecules, and compositions containing the same, that target RNA transcripts (e.g., mRNA) of an ataxin-3 (ATXN3) gene. The disclosure further describes methods for silencing of ATXN3 and the treatment of diseases that may benefit from the silencing of ATXN3 (e.g., spinocerebellar ataxias such as spinocerebellar ataxia type 3) by delivering ATXN3-targeting siRNA molecules to a target tissue of a subject in need.

### BACKGROUND

ATXN3 (ataxin-3) encodes a protein that is implicated in the pathology of diseases including spinocerebellar ataxias. Studies have shown that expansions within the polyglutamine domain in the ataxin-3 protein can cause spinocerebellar ataxias, such as spinocerebellar ataxia type 3 (SCA3). There is a need for therapeutics capable of selectively diminishing ATXN3 activity in a manner that provides effective treatment for spinocerebellar ataxias (e.g., SCA3) or other ATXN3-related diseases or disorders.

### SUMMARY OF THE INVENTION

The present disclosure provides compositions and methods for reduction of ataxin-3 (ATXN3) expression by way of small interfering RNA (siRNA)-mediated silencing of ATXN3 transcripts. The compositions and methods provide the benefit of exhibiting high selectivity toward ATXN3 over other genes.

The siRNA molecules of the disclosure can be used to silence the ATXN3 gene, thereby preventing the translation of the corresponding mRNA transcript and reducing ATXN3 protein expression. This reduction of ATXN3 levels thus prevents disease onset or progression, as expansions in the polyglutamine domain of the ataxin-3 protein is a cause of spinocerebellar ataxias, such as spinocerebellar ataxia type 3 (SCA3). The siRNA molecules of the disclosure can be delivered directly to a subject in need of ATXN3 silencing by way of, for example, injection intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides an siRNA molecule containing an antisense strand and sense strand having complementarity to the antisense strand. The antisense strand has complementarity sufficient to hybridize to a region within an ATXN3 mRNA transcript having the nucleic acid sequence of any one of SEQ ID NOs: 1-192. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 15 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 16 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 17 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 18 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 19 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 20 contiguous nucleobases within the ATXN3 mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 21 contiguous nucleobases within the ATXN3 mRNA transcript having the nucleic acid sequence of any one of SEQ ID NOs: 1-192.

In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the ATXN3 mRNA transcript having the nucleic acid sequence of any one of SEQ ID NOs: 1-192.

In some embodiments, the antisense strand has at least 75% complementarity to the region within the ATXN3 mRNA transcript having the nucleic acid sequence of any one of SEQ ID NOs: 1-192. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the ATXN3 mRNA transcript having the nucleic acid sequence of any one of SEQ ID NOs: 1-192.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the ATXN3 mRNA transcript having the nucleic acid sequence of any one of SEQ ID NOs: 1-192.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the ATXN3 mRNA transcript having the nucleic acid sequence of any one of SEQ ID NOs: 1-192.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the ATXN3 mRNA transcript having the nucleic acid sequence of any one of SEQ ID NOs: 1-192.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the ATXN3 mRNA transcript having the nucleic acid sequence of any one of SEQ ID NOs: 1-192.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the ATXN3 RNA transcript having the nucleic acid sequence of any one of SEQ ID NOs: 1-192.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the ATXN3 mRNA transcript having the nucleic acid sequence of any one of SEQ ID Nos: 1-192.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the ATXN3 mRNA transcript having the nucleic acid sequence of any one of SEQ ID NOs: 1-192.

In some embodiments, the antisense strand has 21 contiguous nucleotides that are fully complementary to a contiguous polynucleotide segment of equal length within the region of the ATXN3 mRNA transcript having the nucleic acid sequence of any one of SEQ ID NOs: 1-192.

In some embodiments, the antisense strand has 9 or fewer nucleotide mismatches relative to a region of 21 contiguous nucleobases of the ATXN3 RNA transcript having the nucleic acid sequence of any one of SEQ ID NOs: 1-192, optionally wherein the antisense strand contains 8 or fewer, 7 or fewer, 6 or fewer, 5 or fewer, 4 or fewer, 3 or fewer, 2 or fewer, or only 1 mismatch relative to the region of the ATXN3 mRNA transcript having the nucleic acid sequence of any one of SEQ ID NOs: 1-192.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-576.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 385-576.

In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 385-576, optionally wherein the antisense strand has a nucleic acid sequence that is at least 96%, 97%, 98%, or 99% identical to the nucleic acid sequence of any one of SEQ ID NOs: 385-576.

In some embodiments, the antisense strand has the nucleic acid sequence of any one of SEQ ID NOs: 385-576.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 193-384.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 193-384, optionally wherein the sense strand has a nucleic acid sequence that is at least 96%, 97%, 98%, or 99% identical to the nucleic acid sequence of any one of SEQ ID NOs: 193-384.

In some embodiments, the siRNA molecule has a sense strand having the nucleic acid sequence of any one of SEQ ID NOs: 193-384.

In some embodiments, the antisense strand has a structure represented by Formula I, wherein Formula I is, in the 5′-to-3′ direction:

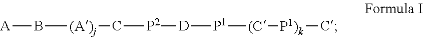

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

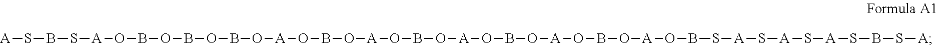

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

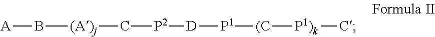

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

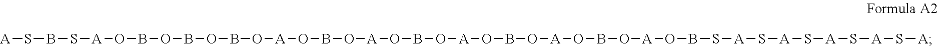

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

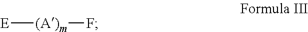

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

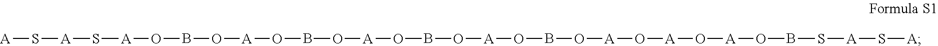

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

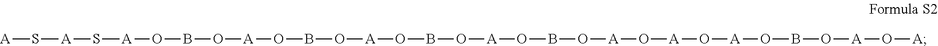

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

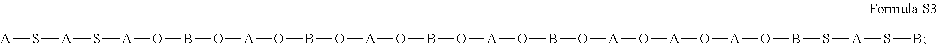

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

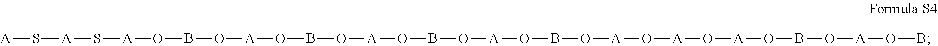

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

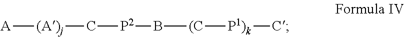

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

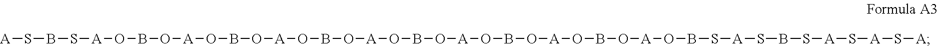

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

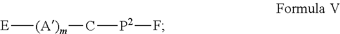

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

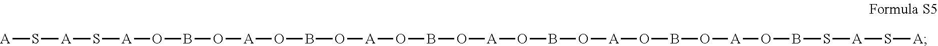

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

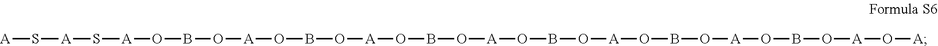

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

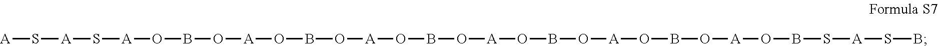

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

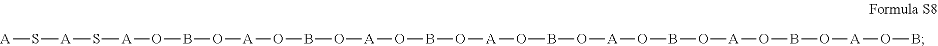

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

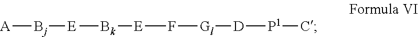

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

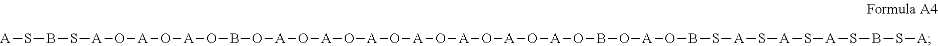

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

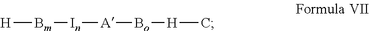

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

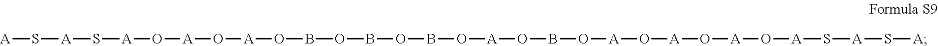

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

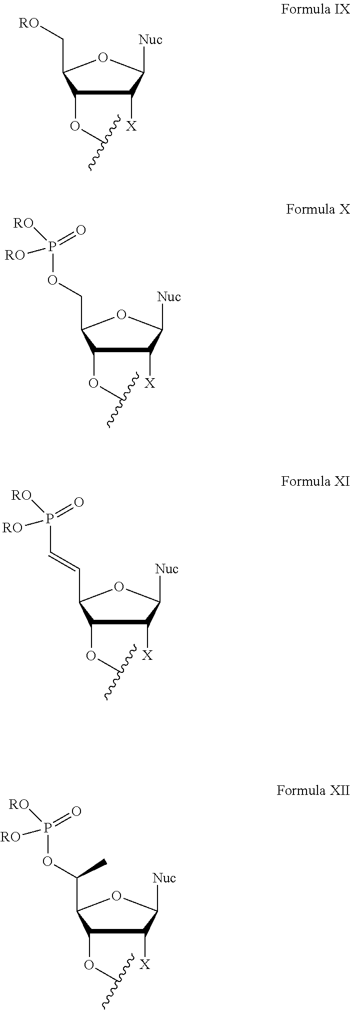

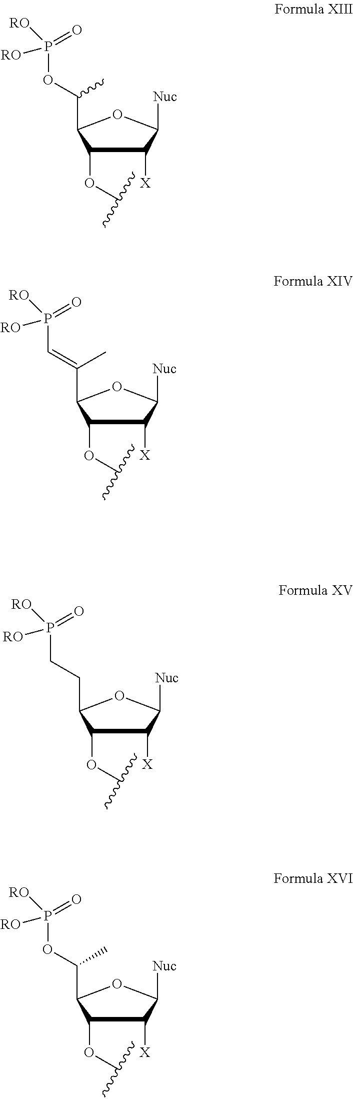

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

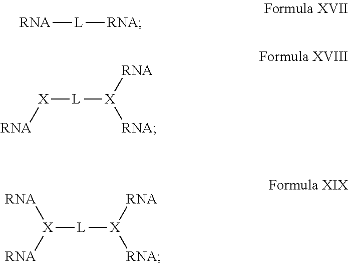

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

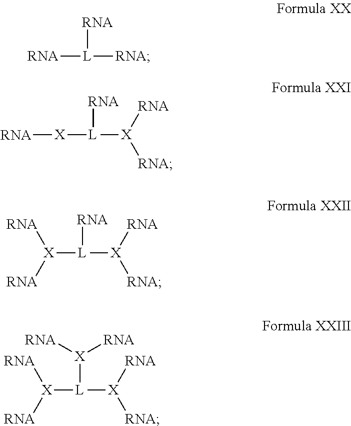

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

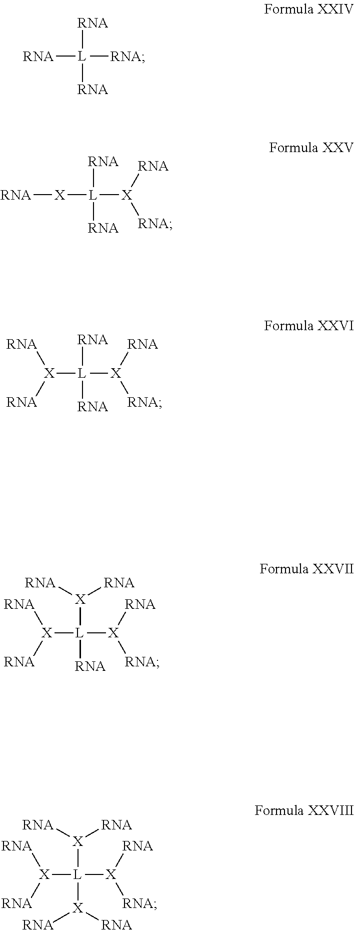

wherein each RNA is, independently, an siRNA molecule, L is a linker, and each X, independently, represents a branch point moiety.

In some embodiments, the tetra-branched siRNA molecule is represented by Formula XXIV.

In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

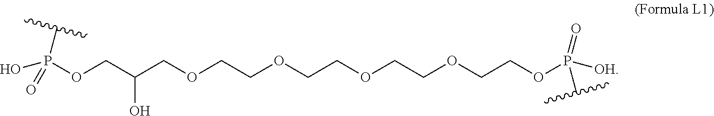 [SMILES: *P(=O)(O)OCCOCCOCCOCCOCC(O)COP(*)(=O)O]

In some embodiments, the linker includes a structure of Formula L2:

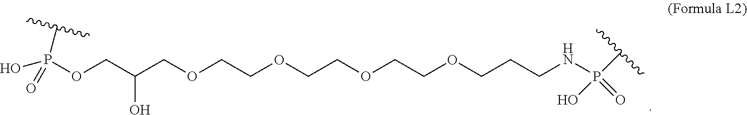 [SMILES: *P(=O)(O)NCCCOCCOCCOCCOCC(O)COP(*)(=O)O]

In some embodiments, the linker includes a structure of Formula L3:

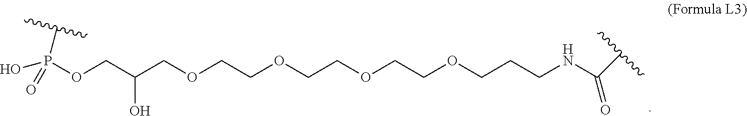 [SMILES: *C(=O)NCCCOCCOCCOCCOCC(O)COP(*)(=O)O]

In some embodiments, the linker includes a structure of Formula L4:

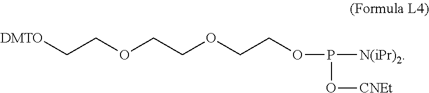

In some embodiments, the linker includes a structure of Formula L5:

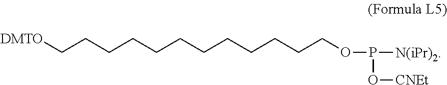

In some embodiments, the linker includes a structure of Formula L6:

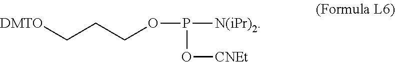

In some embodiments, the linker includes a structure of Formula L7:

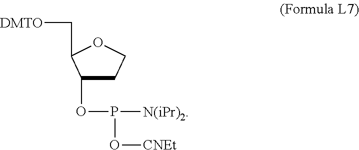

In some embodiments, the linker includes a structure of Formula L8:

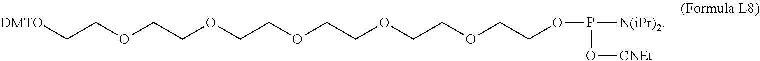

In some embodiments, the linker includes a structure of Formula L9:

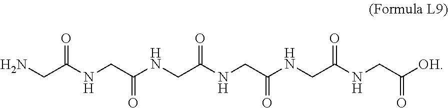 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to a subject diagnosed as having a spinocerebellar ataxia by administering a therapeutically effective amount of the siRNA molecule or a pharmaceutical composition of any of the preceding aspects or embodiments of the disclosure to the subject.

In a further aspect, the disclosure provides a method of treating a spinocerebellar ataxia in a subject in need thereof by administering a therapeutically effective amount of an siRNA molecule or a pharmaceutical composition of any of the preceding aspects or embodiments of the disclosure to the subject.

In some embodiments of the methods described herein, the spinocerebellar ataxia is spinocerebellar ataxia type 3 (SCA3).

In another aspect, the disclosure provides a method of reducing ATXN3 expression in a subject in need thereof by administering a therapeutically effective amount of an siRNA or pharmaceutical composition of any of the preceding aspects or embodiments of the disclosure.

In some embodiments of the methods described herein, the siRNA molecule or the pharmaceutical composition is administered to the subject by way of intracerebroventricular, intrastriatal, intraparenchymal or intrathecal injection. In some embodiments, the siRNA molecule or the pharmaceutical composition is administered to the subject by way of intravenous, intramuscular, or subcutaneous injection.

In some embodiments of any of the methods described herein, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of ATXN3, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂.

In some embodiments, alkenyl may be substituted. Suitable substituents that may be introduced into an alkenyl group include, for example, hydroxy, alkoxy, amino, alkylamino, and halo, among others. As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl —CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that experiences a spinocerebellar ataxia (e.g., spinocerebellar ataxia type 3) and/or contains a gain-of-function ATXN3 variant allele or a repeat expansion of the ATXN3 gene.

As used herein, the term “ATXN3” refers to the gene encoding ataxin-3, including any native ATXN3 gene from any source. The term encompasses “full-length,” unprocessed ATXN3 as well as any form of ATXN3 that results from processing in the cell. The term also encompasses naturally occurring variants of ATXN3, e.g., splice variants or allelic variants. The nucleic acid sequence of an exemplary ATXN3 gene is shown in European Nucleotide Archive (ENA) Accession No. EU009923.1. The amino acid sequence of an exemplary protein encoded by an ATXN3 gene is shown in UNIPROT™ Accession No. P54252.

As used herein, the terms “neuroinflammatory disease” and “neuroinflammatory disorder” are used interchangeably to refer to any condition that is in some way caused by neuroinflammation. “Neuroinflammation” refers to a range of immune responses in the central nervous system (e.g., in microglia). Neuroinflammation may be brain-derived or result from a systemic inflammatory response.

As used herein, the terms “neurodegenerative disease” and “neurodegenerative disorder” are used interchangeably to refer to any condition that is in some way caused by a loss of function or death of cells of the central nervous system or peripheral nervous system. Exemplary neurodegenerative diseases are Alzheimer's disease, Huntington's disease, amyotrophic lateral sclerosis, frontotemporal dementia, and spinocerebellar ataxias.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral (e.g., depressive behavior or apathy) parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, a spinocerebellar ataxia such as spinocerebellar ataxia type 3 (SCA3). For example, clinical benefits in the context of a subject having SCA3 administered an siRNA molecule or siRNA composition of the disclosure include, without limitation, a reduction in symptoms of ataxia, dystonia, or spasticity. “Benefit” and “response” are also used interchangeably to refer to, for example, a reduction in wild type ATXN3 transcripts, mutant ATXN3 transcripts, variant ATXN3 transcripts, splice isoforms of ATXN3 transcripts, and/or overexpressed ATXN3 transcripts.

### DETAILED DESCRIPTION

The present disclosure provides compositions of small interfering RNA (siRNA) molecules with sequence homology to an ataxin-3 (ATXN3) gene and methods for administering said siRNA molecules to a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of ATXN3, relative to other human genes.

The siRNA molecules of the disclosure may feature an antisense strand having a nucleic acid sequence that is complementary to a region of a ATXN3 mRNA transcript having the nucleic acid sequence of any one of SEQ ID NOs: 1-192. The degree of complementarity of the antisense strand to the region of the ATXN3 mRNA transcript may be sufficient for the antisense strand to anneal over the full length of the region of the ATXN3 mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the ATXN3 mRNA transcript.

In some embodiments, the siRNA molecules of the disclosure feature an antisense strand having the nucleic acid sequence of any one of SEQ ID NOs: 385-576, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-576.

In some embodiments, the siRNA molecules of the disclosure feature a sense strand having the nucleic acid sequence of any one of SEQ ID NOs: 193-384, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384.

Exemplary siRNA molecules of the disclosure are those shown in Table 1, below. Table 1 summarizes the antisense strands, sense strands, and corresponding regions of a ATXN3 mRNA transcript that are targeted by each antisense strand.

TABLE 1
Nucleotide sequences for gene-specific ATXN3-targeting siRNA

| Gene | | | | | |
|---|---|---|---|---|---|
| region | | Sense | | | |
| SEQ | | SEQ | | Antisense | |
| ID | | ID | | SEQ ID | |
| NO: | Gene Region | NO: | Sense Sequence | NO: | Antisense Sequence |
| 1 | AAACAAGAAGGC | 193 | AGAAGGCUCACU | 385 | UCAAAGUGAGCCUUC |
| | UCACUUUGU | | UUGA | | UUGUUU |
| 2 | AACAAGAAGGCU | 194 | GAAGGCUCACUU | 386 | UACAAAGUGAGCCUU |
| | CACUUUGUG | | UGUA | | CUUGUU |
| 3 | ACAAGAAGGCU | 195 | AAGGCUCACUUU | 387 | UCACAAAGUGAGCCU |
| | CACUUUGUGC | | GUGA | | UCUUGU |
| 4 | CAAGAAGGCUC | 196 | AGGCUCACUUUG | 388 | UGCACAAAGUGAGCC |
| | ACUUUGUGCU | | UGCA | | UUCUUG |
| 5 | AGAAGGCUCAC | 197 | GCUCACUUUGUG | 389 | UGAGCACAAAGUGAG |
| | UUUGUGCUCA | | CUCA | | CCUUCU |
| 6 | CACUUUGUGCU | 198 | UGUGCUCAACAU | 390 | UGCAAUGUUGAGCAC |
| | CAACAUUGCC | | UGCA | | AAAGUG |
| 7 | GCUCAACAUUG | 199 | ACAUUGCCUGAA | 391 | UUUAUUCAGGCAAUG |
| | CCUGAAUAAC | | UAAA | | UUGAGC |
| 8 | UAACUUAUUGCA | 200 | UAUUGCAAGGAG | 392 | UAUUCUCCUUGCAAU |
| | AGGAGAAUA | | AAUA | | AAGUUA |
| 9 | UUUUAGCCCUG | 201 | GCCCUGUGGAAU | 393 | UAUAAUUCCACAGGG |
| | UGGAAUUAUC | | UAUA | | CUAAAA |
| 10 | UUUAGCCCUGU | 202 | CCCUGUGGAAUU | 394 | UGAUAAUUCCACAGG |
| | GGAAUUAUCC | | AUCA | | GCUAAA |
| 11 | UAGCCCUGUGG | 203 | CUGUGGAAUUAU | 395 | UAGGAUAAUUCCACA |
| | AAUUAUCCUC | | CCUA | | GGGCUA |
| 12 | GUGGAAUUAUC | 204 | AUUAUCCUCAAUU | 396 | UGCAAUUGAGGAUAA |
| | CUCAAUUGCA | | GCA | | UUCCAC |
| 13 | GGAAUUAUCCU | 205 | UAUCCUCAAUUG | 397 | UGUGCAAUUGAGGAU |
| | CAAUUGCACA | | CACA | | AAUUCC |
| 14 | GAUGAGGAGGA | 206 | GGAGGAGAGGAU | 398 | UCUCAUCCUCUCCUC |
| | GAGGAUGAGA | | GAGA | | CUCAUC |
| 15 | AUGGCAGAAGG | 207 | AGAAGGAGGAGU | 399 | UGUAACUCCUCCUUC |
| | AGGAGUUACU | | UACA | | UGCCAU |
| 16 | GCAGAAGGAGG | 208 | AGGAGGAGUUAC | 400 | UCUAGUAACUCCUCC |
| | AGUUACUAGU | | UAGA | | UUCUGC |
| 17 | GAAGGAGGAGU | 209 | AGGAGUUACUAG | 401 | UUCACUAGUAACUCC |
| | UACUAGUGAA | | UGAA | | UCCUUC |
| 18 | GAGUUACUAGU | 210 | ACUAGUGAAGAU | 402 | UAUAAUCUUCACUAG |
| | GAAGAUUAUC | | UAUA | | UAACUC |
| 19 | AGUUACUAGUG | 211 | CUAGUGAAGAUU | 403 | UGAUAAUCUUCACUA |
| | AAGAUUAUCG | | AUCA | | GUAACU |
| 20 | UUACUAGUGAA | 212 | AGUGAAGAUUAU | 404 | UGCGAUAAUCUUCAC |
| | GAUUAUCGCA | | CGCA | | UAGUAA |
| 21 | UUAUCGCACGU | 213 | GCACGUUUUUAC | 405 | UGCUGUAAAAACGUG |
| | UUUUACAGCA | | AGCA | | CGAUAA |
| 22 | CGUUUUUACAG | 214 | UUACAGCAGCCU | 406 | UAGAAGGCUGCUGUA |
| | CAGCCUUCUG | | UCUA | | AAAACG |
| 23 | GUUUUUACAGC | 215 | UACAGCAGCCUU | 407 | UCAGAAGGCUGCUGU |
| | AGCCUUCUGG | | CUGA | | AAAAAC |
| 24 | UUUUUACAGCA | 216 | ACAGCAGCCUUC | 408 | UCCAGAAGGCUGCUG |
| | GCCUUCUGGA | | UGGA | | UAAAAA |
| 25 | ACAGCAGCCUU | 217 | AGCCUUCUGGAA | 409 | UUAUUUCCAGAAGGC |
| | CUGGAAAUAU | | AUAA | | UGCUGU |
| 26 | GCAGCCUUCUG | 218 | CUUCUGGAAAUA | 410 | UCCAUAUUUCCAGAA |
| | GAAAUAUGGA | | UGGA | | GGCUGC |
| 27 | AGCCUUCUGGA | 219 | UCUGGAAAUAUG | 411 | UAUCCAUAUUUCCAG |
| | AAUAUGGAUG | | GAUA | | AAGGCU |
| 28 | UUCUGGAAAUA | 220 | GAAAUAUGGAUG | 412 | UUGUCAUCCAUAUUU |
| | UGGAUGACAG | | ACAA | | CCAGAA |
| 29 | UGGAAAUAUGG | 221 | AUAUGGAUGACA | 413 | UCACUGUCAUCCAUA |
| | AUGACAGUGG | | GUGA | | UUUCCA |
| 30 | UAUGGAUGACA | 222 | AUGACAGUGGUU | 414 | UAAAAACCACUGUCA |
| | GUGGUUUUUU | | UUUA | | UCCAUA |
| 31 | UGACAGUGGUU | 223 | GUGGUUUUUUCU | 415 | UUAGAGAAAAAACCA |
| | UUUUCUCUAU | | CUAA | | CUGUCA |
| 32 | CAGUGGUUUUU | 224 | GUUUUUUCUCUA | 416 | UGAAUAGAGAAAAAA |
| | UCUCUAUUCA | | UUCA | | CCACUG |
| 33 | AGUGGUUUUUU | 225 | UUUUUUCUCUAU | 417 | UUGAAUAGAGAAAAA |
| | CUCUAUUCAG | | UCAA | | ACCACU |
| 34 | GUUUUUUCUCU | 226 | UUCUCUAUUCAG | 418 | UAACCUGAAUAGAGA |
| | AUUCAGGUUA | | GUUA | | AAAAAC |
| 35 | AUUCAGGUUAU | 227 | GGUUAUAAGCAA | 419 | UGCAUUGCUUAUAAC |
| | AAGCAAUGCC | | UGCA | | CUGAAU |
| 36 | UUCAGGUUAUA | 228 | GUUAUAAGCAAU | 420 | UGGCAUUGCUUAUAA |
| | AGCAAUGCCU | | GCCA | | CCUGAA |
| 37 | UCAGGUUAUAA | 229 | UUAUAAGCAAUGC | 421 | UAGGCAUUGCUUAUA |
| | GCAAUGCCUU | | CUA | | ACCUGA |
| 38 | CAGGUUAUAAG | 230 | UAUAAGCAAUGCC | 422 | UAAGGCAUUGCUUAU |
| | CAAUGCCUUG | | UUA | | AACCUG |
| 39 | AGGUUAUAAGC | 231 | AUAAGCAAUGCCU | 423 | UCAAGGCAUUGCUUA |
| | AAUGCCUUGA | | UGA | | UAACCU |
| 40 | UUAUAAGCAAUG | 232 | AGCAAUGCCUUG | 424 | UUUUCAAGGCAUUGC |
| | CCUUGAAAG | | AAAA | | UUAUAA |
| 41 | UAUAAGCAAUGC | 233 | GCAAUGCCUUGA | 425 | UCUUUCAAGGCAUUG |
| | CUUGAAAGU | | AAGA | | CUUAUA |
| 42 | UUUAGAACUAAU | 234 | AACUAAUCCUGUU | 426 | UUGAACAGGAUUAGU |
| | CCUGUUCAA | | CAA | | UCUAAA |
| 43 | UUAGAACUAAUC | 235 | ACUAAUCCUGUU | 427 | UUUGAACAGGAUUAG |
| | CUGUUCAAC | | CAAA | | UUCUAA |
| 44 | UAGAACUAAUCC | 236 | CUAAUCCUGUUC | 428 | UGUUGAACAGGAUUA |
| | UGUUCAACA | | AACA | | GUUCUA |
| 45 | ACUAAUCCUGU | 237 | UCCUGUUCAACA | 429 | UGACUGUUGAACAGG |
| | UCAACAGUCC | | GUCA | | AUUAGU |
| 46 | CUAAUCCUGUU | 238 | CCUGUUCAACAG | 430 | UGGACUGUUGAACAG |
| | CAACAGUCCA | | UCCA | | GAUUAG |
| 47 | CCUGUUCAACA | 239 | UCAACAGUCCAGA | 431 | UACUCUGGACUGUUG |
| | GUCCAGAGUA | | GUA | | AACAGG |
| 48 | CCAGAGUAUCA | 240 | GUAUCAGAGGCU | 432 | UCUGAGCCUCUGAUA |
| | GAGGCUCAGG | | CAGA | | CUCUGG |
| 49 | UAUGCAAUUAUA | 241 | AAUUAUAAGGAAC | 433 | UGUGUUCCUUAUAAU |
| | AGGAACACU | | ACA | | UGCAUA |
| 50 | AUGCAAUUAUAA | 242 | AUUAUAAGGAACA | 434 | UAGUGUUCCUUAUAA |
| | GGAACACUG | | CUA | | UUGCAU |
| 51 | UGCAAUUAUAAG | 243 | UUAUAAGGAACAC | 435 | UCAGUGUUCCUUAUA |
| | GAACACUGG | | UGA | | AUUGCA |
| 52 | CAAUUAUAAGGA | 244 | AUAAGGAACACUG | 436 | UACCAGUGUUCCUUA |
| | ACACUGGUU | | GUA | | UAAUUG |
| 53 | AAUUAUAAGGAA | 245 | UAAGGAACACUG | 437 | UAACCAGUGUUCCUU |
| | CACUGGUUU | | GUUA | | AUAAUU |
| 54 | UUAUAAGGAACA | 246 | AGGAACACUGGU | 438 | UUAAACCAGUGUUCC |
| | CUGGUUUAC | | UUAA | | UUAUAA |
| 55 | UAUAAGGAACAC | 247 | GGAACACUGGUU | 439 | UGUAAACCAGUGUUC |
| | UGGUUUACA | | UACA | | CUUAUA |
| 56 | AUAAGGAACACU | 248 | GAACACUGGUUU | 440 | UUGUAAACCAGUGUU |
| | GGUUUACAG | | ACAA | | CCUUAU |
| 57 | UAAGGAACACU | 249 | AACACUGGUUUA | 441 | UCUGUAAACCAGUGU |
| | GGUUUACAGU | | CAGA | | UCCUUA |
| 58 | AGGAACACUGG | 250 | CACUGGUUUACA | 442 | UAACUGUAAACCAGU |
| | UUUACAGUUA | | GUUA | | GUUCCU |
| 59 | GGAACACUGGU | 251 | ACUGGUUUACAG | 443 | UUAACUGUAAACCAG |
| | UUACAGUUAG | | UUAA | | UGUUCC |
| 60 | GAACACUGGUU | 252 | CUGGUUUACAGU | 444 | UCUAACUGUAAACCA |
| | UACAGUUAGA | | UAGA | | GUGUUC |
| 61 | ACACUGGUUUA | 253 | GGUUUACAGUUA | 445 | UUUCUAACUGUAAAC |
| | CAGUUAGAAA | | GAAA | | CAGUGU |
| 62 | AGAAAAUUAGGA | 254 | AUUAGGAAAACAG | 446 | UCACUGUUUUCCUAA |
| | AAACAGUGG | | UGA | | UUUUCU |
| 63 | GAAAAUUAGGAA | 255 | UUAGGAAAACAGU | 447 | UCCACUGUUUUCCUA |
| | AACAGUGGU | | GGA | | AUUUUC |
| 64 | AAAAUUAGGAAA | 256 | UAGGAAAACAGU | 448 | UACCACUGUUUUCCU |
| | ACAGUGGUU | | GGUA | | AAUUUU |
| 65 | AGGAAAACAGU | 257 | AACAGUGGUUUA | 449 | UAGUUAAACCACUGU |
| | GGUUUAACUU | | ACUA | | UUUCCU |
| 66 | GGAAAACAGUG | 258 | ACAGUGGUUUAA | 450 | UAAGUUAAACCACUG |
| | GUUUAACUUG | | CUUA | | UUUUCC |
| 67 | GAAAACAGUGG | 259 | CAGUGGUUUAAC | 451 | UCAAGUUAAACCACU |
| | UUUAACUUGA | | UUGA | | GUUUUC |
| 68 | AAACAGUGGUU | 260 | GUGGUUUAACUU | 452 | UUUCAAGUUAAACCA |
| | UAACUUGAAU | | GAAA | | CUGUUU |
| 69 | AACAGUGGUUU | 261 | UGGUUUAACUUG | 453 | UAUUCAAGUUAAACC |
| | AACUUGAAUU | | AAUA | | ACUGUU |
| 70 | UCUCUCUUGAC | 262 | CUUGACGGGUCC | 454 | UUCUGGACCCGUCAA |
| | GGGUCCAGAA | | AGAA | | GAGAGA |
| 71 | AUAUCAGAUACA | 263 | AGAUACAUAUCUU | 455 | UGCAAGAUAUGUAUC |
| | UAUCUUGCA | | GCA | | UGAUAU |
| 72 | AUCAGAUACAUA | 264 | AUACAUAUCUUGC | 456 | UGUGCAAGAUAUGUA |
| | UCUUGCACU | | ACA | | UCUGAU |
| 73 | ACUUUUCUUGG | 265 | UCUUGGCUCAAU | 457 | UGUAAUUGAGCCAAG |
| | CUCAAUUACA | | UACA | | AAAAGU |
| 74 | UCUUGGCUCAA | 266 | GCUCAAUUACAAC | 458 | UCUGUUGUAAUUGAG |
| | UUACAACAGG | | AGA | | CCAAGA |
| 75 | CUUGGCUCAAU | 267 | CUCAAUUACAACA | 459 | UCCUGUUGUAAUUGA |
| | UACAACAGGA | | GGA | | GCCAAG |
| 76 | UUGGCUCAAUU | 268 | UCAAUUACAACAG | 460 | UUCCUGUUGUAAUUG |
| | ACAACAGGAA | | GAA | | AGCCAA |
| 77 | UGGCUCAAUUA | 269 | CAAUUACAACAGG | 461 | UUUCCUGUUGUAAUU |
| | CAACAGGAAG | | AAA | | GAGCCA |
| 78 | GGCUCAAUUAC | 270 | AAUUACAACAGGA | 462 | UCUUCCUGUUGUAAU |
| | AACAGGAAGG | | AGA | | UGAGCC |
| 79 | CAAUUACAACAG | 271 | ACAACAGGAAGG | 463 | UUAACCUUCCUGUUG |
| | GAAGGUUAU | | UUAA | | UAAUUG |
| 80 | AAUUACAACAGG | 272 | CAACAGGAAGGU | 464 | UAUAACCUUCCUGUU |
| | AAGGUUAUU | | UAUA | | GUAAUU |
| 81 | CAACAGGAAGG | 273 | GGAAGGUUAUUC | 465 | UAUAGAAUAACCUUC |
| | UUAUUCUAUA | | UAUA | | CUGUUG |
| 82 | ACAGGAAGGUU | 274 | AAGGUUAUUCUA | 466 | UAUAUAGAAUAACCU |
| | AUUCUAUAUU | | UAUA | | UCCUGU |
| 83 | CAGGAAGGUUA | 275 | AGGUUAUUCUAU | 467 | UAAUAUAGAAUAACC |
| | UUCUAUAUUU | | AUUA | | UUCCUG |
| 84 | UCUGCCAGAUU | 276 | CAGAUUGCGAAG | 468 | UCAGCUUCGCAAUCU |
| | GCGAAGCUGA | | CUGA | | GGCAGA |
| 85 | AUGCAUCGACC | 277 | UCGACCAAAACUU | 469 | UAUAAGUUUUGGUCG |
| | AAAACUUAUU | | AUA | | AUGCAU |
| 86 | GCAUCGACCAAA | 278 | GACCAAAACUUAU | 470 | UCAAUAAGUUUUGGU |
| | ACUUAUUGG | | UGA | | CGAUGC |
| 87 | UCGACCAAAACU | 279 | CAAAACUUAUUGG | 471 | UCUCCAAUAAGUUUU |
| | UAUUGGAGA | | AGA | | GGUCGA |
| 88 | CCAAAACUUAUU | 280 | ACUUAUUGGAGA | 472 | UUCUUCUCCAAUAAG |
| | GGAGAAGAA | | AGAA | | UUUUGG |
| 89 | ACUUAUUGGAG | 281 | UUGGAGAAGAAU | 473 | UCUAAUUCUUCUCCA |
| | AAGAAUUAGC | | UAGA | | AUAAGU |
| 90 | UAUUGGAGAAG | 282 | GAGAAGAAUUAG | 474 | UGUGCUAAUUCUUCU |
| | AAUUAGCACA | | CACA | | CCAAUA |
| 91 | AAAGAGCAAAGA | 283 | GCAAAGAGUCCA | 475 | UUUAUGGACUCUUUG |
| | GUCCAUAAA | | UAAA | | CUCUUU |
| 92 | AGAGUCCAUAAA | 284 | CCAUAAAACAGAC | 476 | UAGGUCUGUUUUAUG |
| | ACAGACCUG | | CUA | | GACUCU |
| 93 | UCCAUAAAACAG | 285 | AAAACAGACCUGG | 477 | UUUCCAGGUCUGUUU |
| | ACCUGGAAC | | AAA | | UAUGGA |
| 94 | UAGAAGCAAAUG | 286 | GCAAAUGAUGGC | 478 | UUGAGCCAUCAUUUG |
| | AUGGCUCAG | | UCAA | | CUUCUA |
| 95 | AAUGAUGGCUC | 287 | UGGCUCAGGAAU | 479 | UAACAUUCCUGAGCC |
| | AGGAAUGUUA | | GUUA | | AUCAUU |
| 96 | GCAGAGGGCUC | 288 | GGGCUCUGGCAC | 480 | UUUAGUGCCAGAGCC |
| | UGGCACUAAG | | UAAA | | CUCUGC |
| 97 | CUAAGUCGCCA | 289 | UCGCCAAGAAAUU | 481 | UUCAAUUUCUUGGCG |
| | AGAAAUUGAC | | GAA | | ACUUAG |
| 98 | UUCAGCUAAGU | 290 | CUAAGUAUGCAA | 482 | UACCUUGCAUACUUA |
| | AUGCAAGGUA | | GGUA | | GCUGAA |
| 99 | UCAGCUAAGUA | 291 | UAAGUAUGCAAG | 483 | UUACCUUGCAUACUU |
| | UGCAAGGUAG | | GUAA | | AGCUGA |
| 100 | GCUAAGUAUGC | 292 | GUAUGCAAGGUA | 484 | UAACUACCUUGCAUA |
| | AAGGUAGUUC | | GUUA | | CUUAGC |
| 101 | CUAAGUAUGCAA | 293 | UAUGCAAGGUAG | 485 | UGAACUACCUUGCAU |
| | GGUAGUUCC | | UUCA | | ACUUAG |
| 102 | UAAGUAUGCAA | 294 | AUGCAAGGUAGU | 486 | UGGAACUACCUUGCA |
| | GGUAGUUCCA | | UCCA | | UACUUA |
| 103 | AAGUAUGCAAG | 295 | UGCAAGGUAGUU | 487 | UUGGAACUACCUUGC |
| | GUAGUUCCAG | | CCAA | | AUACUU |
| 104 | AGUAUGCAAGG | 296 | GCAAGGUAGUUC | 488 | UCUGGAACUACCUUG |
| | UAGUUCCAGA | | CAGA | | CAUACU |
| 105 | GUAUGCAAGGU | 297 | CAAGGUAGUUCC | 489 | UUCUGGAACUACCUU |
| | AGUUCCAGAA | | AGAA | | GCAUAC |
| 106 | AUGCAAGGUAG | 298 | AGGUAGUUCCAG | 490 | UUUUCUGGAACUACC |
| | UUCCAGAAAC | | AAAA | | UUGCAU |
| 107 | AUGACACAGACA | 299 | ACAGACAUCAGG | 491 | UGUACCUGAUGUCUG |
| | UCAGGUACA | | UACA | | UGUCAU |
| 108 | GACACAGACAUC | 300 | AGACAUCAGGUA | 492 | UUUGUACCUGAUGUC |
| | AGGUACAAA | | CAAA | | UGUGUC |
| 109 | CACAGACAUCAG | 301 | ACAUCAGGUACAA | 493 | UAUUUGUACCUGAUG |
| | GUACAAAUC | | AUA | | UCUGUG |
| 110 | UCAGGUACAAAU | 302 | UACAAAUCUUACU | 494 | UGAAGUAAGAUUUGU |
| | CUUACUUCA | | UCA | | ACCUGA |
| 111 | UCUUACUUCAG | 303 | CUUCAGAAGAGC | 495 | UGAAGCUCUUCUGAA |
| | AAGAGCUUCG | | UUCA | | GUAAGA |
| 112 | UUACUUCAGAA | 304 | UCAGAAGAGCUU | 496 | UCCGAAGCUCUUCUG |
| | GAGCUUCGGA | | CGGA | | AAGUAA |
| 113 | UACUUCAGAAGA | 305 | CAGAAGAGCUUC | 497 | UUCCGAAGCUCUUCU |
| | GCUUCGGAA | | GGAA | | GAAGUA |
| 114 | UCAGAAGAGCU | 306 | AGAGCUUCGGAA | 498 | UCUCUUCCGAAGCUC |
| | UCGGAAGAGA | | GAGA | | UUCUGA |
| 115 | GCUUCGGAAGA | 307 | GGAAGAGACGAG | 499 | UCUUCUCGUCUCUUC |
| | GACGAGAAGC | | AAGA | | CGAAGC |
| 116 | CUUCGGAAGAG | 308 | GAAGAGACGAGA | 500 | UGCUUCUCGUCUCUU |
| | ACGAGAAGCC | | AGCA | | CCGAAG |
| 117 | GGAAGAGACGA | 309 | AGACGAGAAGCC | 501 | UGUAGGCUUCUCGUC |
| | GAAGCCUACU | | UACA | | UCUUCC |
| 118 | GAAGAGACGAG | 310 | GACGAGAAGCCU | 502 | UAGUAGGCUUCUCGU |
| | AAGCCUACUU | | ACUA | | CUCUUC |
| 119 | AAGAGACGAGAA | 311 | ACGAGAAGCCUA | 503 | UAAGUAGGCUUCUCG |
| | GCCUACUUU | | CUUA | | UCUCUU |
| 120 | AGAGACGAGAA | 312 | CGAGAAGCCUAC | 504 | UAAAGUAGGCUUCUC |
| | GCCUACUUUG | | UUUA | | GUCUCU |
| 121 | GAGACGAGAAG | 313 | GAGAAGCCUACU | 505 | UCAAAGUAGGCUUCU |
| | CCUACUUUGA | | UUGA | | CGUCUC |
| 122 | AGACGAGAAGC | 314 | AGAAGCCUACUU | 506 | UUCAAAGUAGGCUUC |
| | CUACUUUGAA | | UGAA | | UCGUCU |
| 123 | AGAAGCCUACU | 315 | CCUACUUUGAAAA | 507 | UGUUUUUCAAAGUAG |
| | UUGAAAAACA | | ACA | | GCUUCU |
| 124 | ACUUGGGAGUG | 316 | GGAGUGAUCUAG | 508 | UCACCUAGAUCACUC |
| | AUCUAGGUGA | | GUGA | | CCAAGU |
| 125 | CUUGGGAGUGA | 317 | GAGUGAUCUAGG | 509 | UUCACCUAGAUCACU |
| | UCUAGGUGAU | | UGAA | | CCCAAG |
| 126 | UUGGGAGUGAU | 318 | AGUGAUCUAGGU | 510 | UAUCACCUAGAUCAC |
| | CUAGGUGAUG | | GAUA | | UCCCAA |
| 127 | GUGAUCUAGGU | 319 | CUAGGUGAUGCU | 511 | UCAUAGCAUCACCUA |
| | GAUGCUAUGA | | AUGA | | GAUCAC |
| 128 | CAGGCAGCUGU | 320 | AGCUGUGACCAU | 512 | UGACAUGGUCACAGC |
| | GACCAUGUCU | | GUCA | | UGCCUG |
| 129 | GCAGCUGUGAC | 321 | UGUGACCAUGUC | 513 | UAAAGACAUGGUCAC |
| | CAUGUCUUUA | | UUUA | | AGCUGC |
| 130 | CAGCUGUGACC | 322 | GUGACCAUGUCU | 514 | UUAAAGACAUGGUCA |
| | AUGUCUUUAG | | UUAA | | CAGCUG |
| 131 | AGCUGUGACCA | 323 | UGACCAUGUCUU | 515 | UCUAAAGACAUGGUC |
| | UGUCUUUAGA | | UAGA | | ACAGCU |
| 132 | GCUGUGACCAU | 324 | GACCAUGUCUUU | 516 | UUCUAAAGACAUGGU |
| | GUCUUUAGAA | | AGAA | | CACAGC |
| 133 | CUGUGACCAUG | 325 | ACCAUGUCUUUA | 517 | UUUCUAAAGACAUGG |
| | UCUUUAGAAA | | GAAA | | UCACAG |
| 134 | GACCAUGUCUU | 326 | UGUCUUUAGAAA | 518 | UCAGUUUCUAAAGAC |
| | UAGAAACUGU | | CUGA | | AUGGUC |
| 135 | CCAUGUCUUUA | 327 | UCUUUAGAAACU | 519 | UGACAGUUUCUAAAG |
| | GAAACUGUCA | | GUCA | | ACAUGG |
| 136 | UGUCUUUAGAA | 328 | UUAGAAACUGUCA | 520 | UUCUGACAGUUUCUA |
| | ACUGUCAGAA | | GAA | | AAGACA |
| 137 | AACUGUCAGAAA | 329 | UCAGAAAUGAUUU | 521 | UUCAAAUCAUUUCUG |
| | UGAUUUGAA | | GAA | | ACAGUU |
| 138 | AUCCUGUGUGA | 330 | GUGUGAUUACAG | 522 | UAUGCUGUAAUCACA |
| | UUACAGCAUA | | CAUA | | CAGGAU |
| 139 | UCCUGUGUGAU | 331 | UGUGAUUACAGC | 523 | UUAUGCUGUAAUCAC |
| | UACAGCAUAG | | AUAA | | ACAGGA |
| 140 | CCUGUGUGAUU | 332 | GUGAUUACAGCA | 524 | UCUAUGCUGUAAUCA |
| | ACAGCAUAGG | | UAGA | | CACAGG |
| 141 | UACAGCAUAGG | 333 | CAUAGGGUCCAC | 525 | UAAAGUGGACCCUAU |
| | GUCCACUUUG | | UUUA | | GCUGUA |
| 142 | AGGGUCCACUU | 334 | CCACUUUGGUAA | 526 | UACAUUACCAAAGUG |
| | UGGUAAUGUG | | UGUA | | GACCCU |
| 143 | GUCCACUUUGG | 335 | CUUUGGUAAUGU | 527 | UGACACAUUACCAAA |
| | UAAUGUGUCA | | GUCA | | GUGGAC |
| 144 | UCCACUUUGGU | 336 | UUUGGUAAUGUG | 528 | UUGACACAUUACCAA |
| | AAUGUGUCAA | | UCAA | | AGUGGA |
| 145 | CCACUUUGGUA | 337 | UUGGUAAUGUGU | 529 | UUUGACACAUUACCA |
| | AUGUGUCAAA | | CAAA | | AAGUGG |
| 146 | UUUGGUAAUGU | 338 | UAAUGUGUCAAA | 530 | UCUCUUUGACACAUU |
| | GUCAAAGAGA | | GAGA | | ACCAAA |
| 147 | GUAAUGUGUCA | 339 | GUGUCAAAGAGA | 531 | UUCAUCUCUUUGACA |
| | AAGAGAUGAG | | UGAA | | CAUUAC |
| 148 | UAAUGUGUCAAA | 340 | UGUCAAAGAGAU | 532 | UCUCAUCUCUUUGAC |
| | GAGAUGAGG | | GAGA | | ACAUUA |
| 149 | UAGCGGUUUGC | 341 | GUUUGCAAACAAA | 533 | UAUUUUGUUUGCAAA |
| | AAACAAAAUG | | AUA | | CCGCUA |
| 150 | GUUUGCAAACAA | 342 | CAAACAAAAUGAU | 534 | UCCAUCAUUUUGUUU |
| | AAUGAUGGG | | GGA | | GCAAAC |
| 151 | UUUGCAAACAAA | 343 | AAACAAAAUGAUG | 535 | UCCCAUCAUUUUGUU |
| | AUGAUGGGA | | GGA | | UGCAAA |
| 152 | GUCGGUUGUAG | 344 | UUGUAGGACUAA | 536 | UUAUUUAGUCCUACA |
| | GACUAAAUAA | | AUAA | | ACCGAC |
| 153 | GUUGUAGGACU | 345 | AGGACUAAAUAAU | 537 | UUCAUUAUUUAGUCC |
| | AAAUAAUGAU | | GAA | | UACAAC |
| 154 | GUAGGACUAAA | 346 | ACUAAAUAAUGAU | 538 | UAGAUCAUUAUUUAG |
| | UAAUGAUCUU | | CUA | | UCCUAC |
| 155 | AGGACUAAAUAA | 347 | UAAAUAAUGAUCU | 539 | UGAAGAUCAUUAUUU |
| | UGAUCUUCC | | UCA | | AGUCCU |
| 156 | UCCAAAUAUUAG | 348 | AUAUUAGCCAAAG | 540 | UCUCUUUGGCUAAUA |
| | CCAAAGAGG | | AGA | | UUUGGA |
| 157 | AAAUAUUAGCCA | 349 | UUAGCCAAAGAG | 541 | UUGCCUCUUUGGCUA |
| | AAGAGGCAU | | GCAA | | AUAUUU |
| 158 | AUUAGCCAAAGA | 350 | CCAAAGAGGCAU | 542 | UUGAAUGCCUCUUUG |
| | GGCAUUCAG | | UCAA | | GCUAAU |
| 159 | UUAGCCAAAGA | 351 | CAAAGAGGCAUU | 543 | UCUGAAUGCCUCUUU |
| | GGCAUUCAGC | | CAGA | | GGCUAA |
| 160 | GCCAAAGAGGC | 352 | AGAGGCAUUCAG | 544 | UUUGCUGAAUGCCUC |
| | AUUCAGCAAU | | CAAA | | UUUGGC |
| 161 | AAGAGGCAUUC | 353 | GCAUUCAGCAAU | 545 | UUUAAUUGCUGAAUG |
| | AGCAAUUAAA | | UAAA | | CCUCUU |
| 162 | AGAGGCAUUCA | 354 | CAUUCAGCAAUUA | 546 | UUUUAAUUGCUGAAU |
| | GCAAUUAAAG | | AAA | | GCCUCU |
| 163 | GGCAUUCAGCA | 355 | UCAGCAAUUAAAG | 547 | UGUCUUUAAUUGCUG |
| | AUUAAAGACA | | ACA | | AAUGCC |
| 164 | CUUUUUCUUUU | 356 | UCUUUUUUGAGU | 548 | UCACACUCAAAAAAG |
| | UUGAGUGUGC | | GUGA | | AAAAAG |
| 165 | UCUUUUUUGAG | 357 | UUUGAGUGUGCA | 549 | UUAUUGCACACUCAA |
| | UGUGCAAUAU | | AUAA | | AAAAGA |
| 166 | CUUUUUUGAGU | 358 | UUGAGUGUGCAA | 550 | UAUAUUGCACACUCA |
| | GUGCAAUAUG | | UAUA | | AAAAAG |
| 167 | UUAGGGCAUUU | 359 | GCAUUUUUCUUG | 551 | UAUCCAAGAAAAAUG |
| | UUCUUGGAUC | | GAUA | | CCCUAA |
| 168 | CCAAAUUAACCU | 360 | UUAACCUUUCAG | 552 | UUUCCUGAAAGGUUA |
| | UUCAGGAAA | | GAAA | | AUUUGG |
| 169 | UUAACCUUUCA | 361 | CUUUCAGGAAAG | 553 | UAUACUUUCCUGAAA |
| | GGAAAGUAUC | | UAUA | | GGUUAA |
| 170 | UAACCUUUCAG | 362 | UUUCAGGAAAGU | 554 | UGAUACUUUCCUGAA |
| | GAAAGUAUCU | | AUCA | | AGGUUA |
| 171 | GAUGUUGAUAA | 363 | UGAUAAUAGUAAU | 555 | UCCAUUACUAUUAUC |
| | UAGUAAUGGU | | GGA | | AACAUC |
| 172 | GAUAAUAGUAAU | 364 | UAGUAAUGGUUC | 556 | UCUAGAACCAUUACU |
| | GGUUCUAGA | | UAGA | | AUUAUC |
| 173 | UUCUCCCUUCA | 365 | CCUUCAACUGUA | 557 | UGUAUACAGUUGAAG |
| | ACUGUAUACC | | UACA | | GGAGAA |
| 174 | CCGUGUGCUCC | 366 | UGCUCCAGUGUU | 558 | UGAAAACACUGGAGC |
| | AGUGUUUUCU | | UUCA | | ACACGG |
| 175 | UCAUUAUUUUC | 367 | AUUUUCCCACAAU | 559 | UGAAUUGUGGGAAAA |
| | CCACAAUUCU | | UCA | | UAAUGA |
| 176 | UUUCCCACAAUU | 368 | CACAAUUCUUUU | 560 | UUUCAAAAGAAUUGU |
| | CUUUUGAAA | | GAAA | | GGGAAA |
| 177 | UCCCACAAUUCU | 369 | CAAUUCUUUUGAA | 561 | UCUUUCAAAAGAAUU |
| | UUUGAAAGA | | AGA | | GUGGGA |
| 178 | UGAAAGAUGGU | 370 | GAUGGUAAUCUU | 562 | UGAAAAGAUUACCAU |
| | AAUCUUUUCU | | UUCA | | CUUUCA |
| 179 | CCCUACGAUGG | 371 | CGAUGGGAUCAU | 563 | UAUAAUGAUCCCAUC |
| | GAUCAUUAUU | | UAUA | | GUAGGG |
| 180 | CGAUGGGAUCA | 372 | GGAUCAUUAUUU | 564 | UAUGAAAUAAUGAUC |
| | UUAUUUCAUG | | CAUA | | CCAUCG |
| 181 | UGGGAUCAUUA | 373 | UCAUUAUUUCAU | 565 | UGUCAUGAAAUAAUG |
| | UUUCAUGACU | | GACA | | AUCCCA |
| 182 | AGUACUUGAGA | 374 | UUGAGAAUAAAUG | 566 | UCUCAUUUAUUCUCA |
| | AUAAAUGAGC | | AGA | | AGUACU |
| 183 | UCUAGGUAAGG | 375 | GUAAGGCCUGCU | 567 | UGUGAGCAGGCCUUA |
| | CCUGCUCACC | | CACA | | CCUAGA |
| 184 | UUCAUCAUGUU | 376 | CAUGUUCGCUAC | 568 | UAAGGUAGCGAACAU |
| | CGCUACCUUC | | CUUA | | GAUGAA |
| 185 | AUCAUGUUCGC | 377 | GUUCGCUACCUU | 569 | UGUGAAGGUAGCGAA |
| | UACCUUCACA | | CACA | | CAUGAU |
| 186 | UCAUGUUCGCU | 378 | UUCGCUACCUUC | 570 | UUGUGAAGGUAGCGA |
| | ACCUUCACAC | | ACAA | | ACAUGA |
| 187 | UGUUCGCUACC | 379 | GCUACCUUCACA | 571 | UAAGUGUGAAGGUAG |
| | UUCACACUUU | | CUUA | | CGAACA |
| 188 | UUCGCUACCUU | 380 | UACCUUCACACUU | 572 | UUAAAGUGUGAAGGU |
| | CACACUUUAU | | UAA | | AGCGAA |
| 189 | UUUAUCUGACA | 381 | CUGACAUACGAG | 573 | UGAGCUCGUAUGUCA |
| | UACGAGCUCC | | CUCA | | GAUAAA |
| 190 | AGCUCCAUGUG | 382 | CAUGUGAUUUUU | 574 | UAGCAAAAAUCACAU |
| | AUUUUUGCUU | | GCUA | | GGAGCU |
| 191 | GCUCCAUGUGA | 383 | AUGUGAUUUUUG | 575 | UAAGCAAAAAUCACA |
| | UUUUUGCUUU | | CUUA | | UGGAGC |
| 192 | UAAAUUUCCCU | 384 | UUCCCUGGAUUA | 576 | UCCUUAAUCCAGGGA |
| | GGAUUAAGGA | | AGGA | | AAUUUA |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100%70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

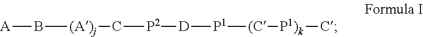

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

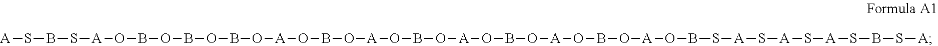

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

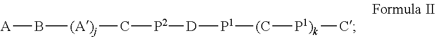

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

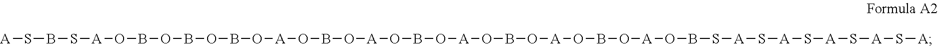

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

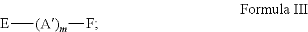

wherein E is represented by the formula (C—P¹)₂; F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

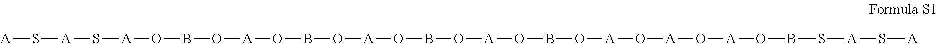

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

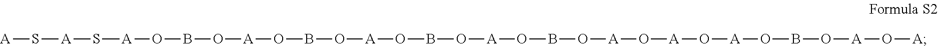

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

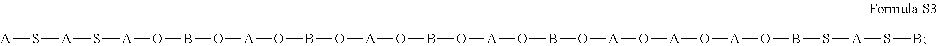

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

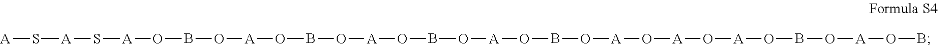

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

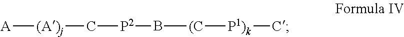

wherein A is represented by the formula C—P¹-D-P¹; each A′ is represented by the formula C—P²-D-P²; B is represented by the formula D-P¹—C—P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

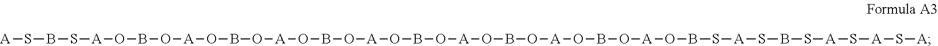

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

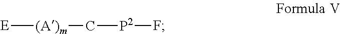

wherein E is represented by the formula (C—P¹)₂; F is represented by the formula D-P¹—C—P¹—C, D-P²—C—P²—C, D-P¹—C—P¹-D, or D-P²—C—P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

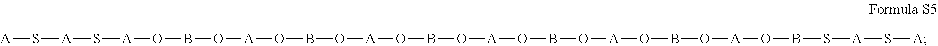

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

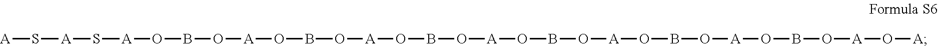

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

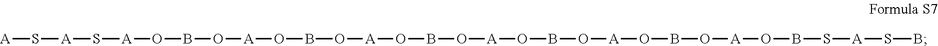

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

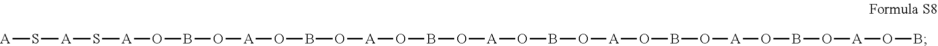

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

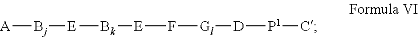

wherein A is represented by the formula C—P¹-D-P¹; each B is represented by the formula C—P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²—C—P²; F is represented by the formula D-P¹—C—P¹; each G is represented by the formula C—P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

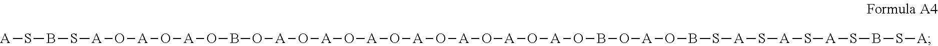

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

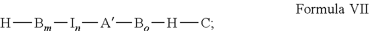

wherein A′ is represented by the formula C—P²-D-P²; each H is represented by the formula (C—P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

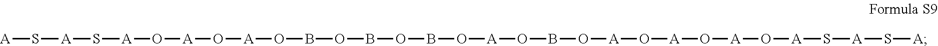

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

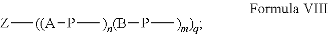

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
   Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

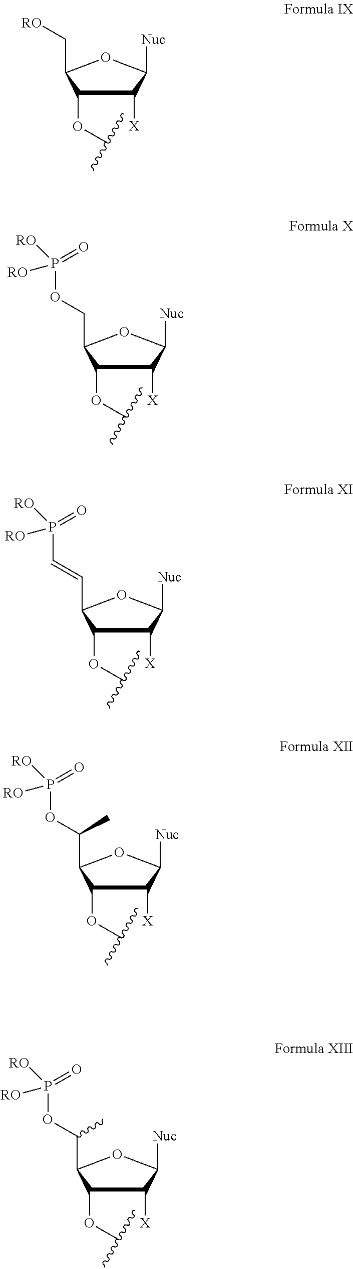

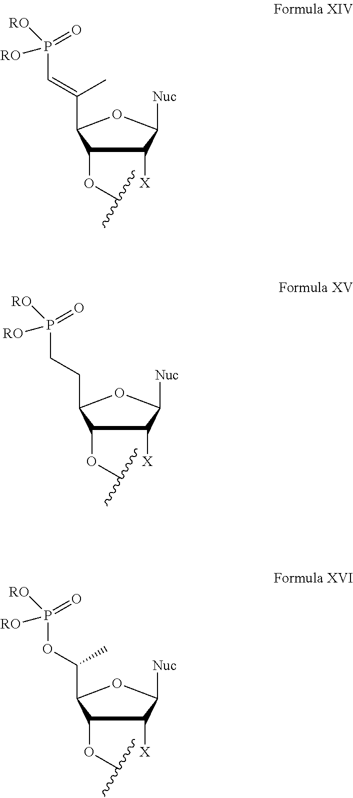

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA—L—RNA Formula XVII | 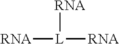 Formula XX | 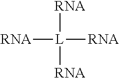 Formula XXIV |
| 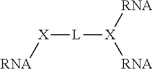 Formula XVIII | 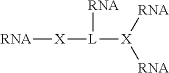 Formula XXI | 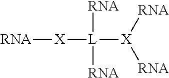 Formula XXV |
| 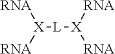 Formula XIX | 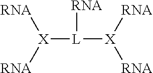 Formula XXII | 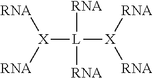 Formula XXVI |
| | 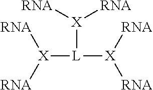 Formula XXIII | 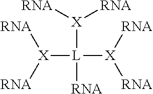 Formula XXVII |
| | | 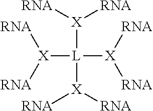 Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

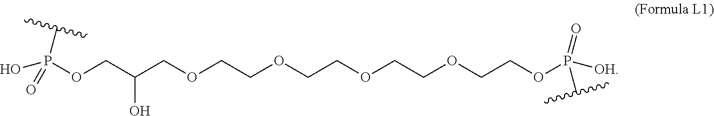 [SMILES: *P(=O)(O)OCCOCCOCCOCCOCC(O)COP(*)(=O)O]

In some embodiments, the linker has a structure of Formula L2:

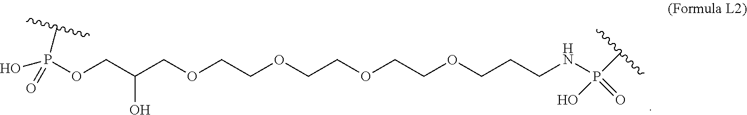 [SMILES: *P(=O)(O)NCCCOCCOCCOCCOCC(O)COP(*)(=O)O]

In some embodiments, the linker has a structure of Formula L3:

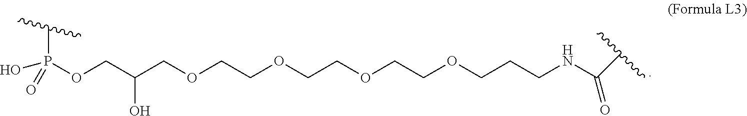 [SMILES: *C(=O)NCCCOCCOCCOCCOCC(O)COP(*)(=O)O]

In some embodiments, the linker has a structure of Formula L4:

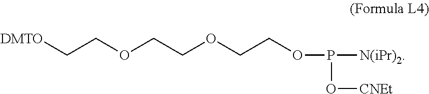

In some embodiments, the linker has a structure of Formula L5:

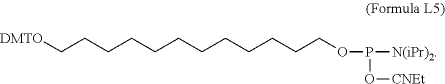

In some embodiments, the linker has a structure of Formula L6:

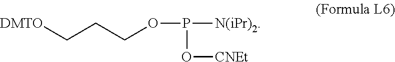

In some embodiments, the linker has a structure of Formula L7, as is shown below:

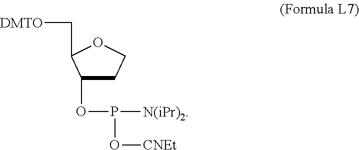

In some embodiments, the linker has a structure of Formula L8:

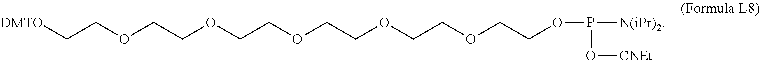

In some embodiments, the linker has a structure of Formula L9:

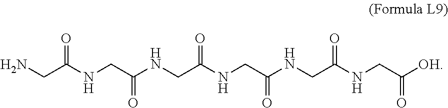 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The ATXN3-targeting siRNA molecules of the disclosure may be delivered to a subject, thereby treating a spinocerebellar ataxia, such as spinocerebellar ataxia type 3 (SCA3), and/or mitigating phenotypes associated with the disorders. For example, the siRNA molecules may be delivered to a subject, thereby treating SCA3 and/or mitigating disease associated phenotypes (e.g., ataxia, dystonia, or spasticity). Furthermore, the siRNA molecules of the disclosure may also be delivered to a subject having a variant of the ATXN3 gene for which siRNA-mediated gene silencing of the ATXN3 variant gene reduces the expression level of ATXN3 transcript, thereby treating SCA3 or other ATXN3-related diseases or disorders.

The disclosure provides methods of treating a subject by way of ATXN3 gene silencing with one or more of the siRNA molecules described herein. The gene silencing may be performed in a subject to silence wild type ATXN3 transcripts, mutant ATXN3 transcripts, splice isoforms of ATXN3 transcripts, and/or overexpressed ATXN3 transcripts thereof, relative to a healthy subject. The method may include delivering to the CNS or affected tissues of the subject (e.g., a human) the siRNA molecules of the disclosure or a pharmaceutical composition containing the same by any appropriate route of administration (e.g., intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, a spinocerebellar ataxia (e.g., SCA3) and/or any other medical risk(s) associated with repeat expansions or a gain of function mutations in the ATXN3 gene. Subjects that may be treated with the siRNA molecules disclosed herein may include, for example, humans, monkeys, rats, mice, pigs, and other mammals containing at least one orthologous copy of the ATXN3 gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. ATXN3 Knockdown

### Objective

This Example describes the results of a series of experiments undertaken to investigate the ability of siRNA molecules complementary to specific regions within a human ATXN3 mRNA transcript to effectuate reduced expression of the ATXN3 gene.

### Materials and Methods

MeWo cells were actively transfected with ATXN3 siRNA at either 20 nM or 0.2 nM concentration. After 72 hours, cells were lysed, and mRNA levels of ATXN3 and a housekeeping gene (ATP5B) were assessed via reverse transcription-quantitative polymerase chain reaction (RT-qPCR), using standard reagents and Applied Biosystems TaqMan Assays. Results are presented as the percent residual ATXN3 mRNA relative to untreated control cells in the same assay.

### Results

Cells were treated with an siRNA molecule of the disclosure having an antisense strand and a sense strand as shown in Tables 3 and 4, below. The knockdown efficiency of the siRNA molecule was measured as the residual mRNA expression as a percent of untreated at 20 nM and 0.2 nM. The knockdown results are reported in Table 3 (20 nM) and Table 4 (0.2 nM).

TABLE 3
ATXN3 knockdown with siRNA molecules
of the disclosure at 20 nM

| | | Residual |
|---|---|---|
| | | mRNA |
| | | expression |
| Sense | Antisense | (% of |
| SEQ ID | SEQ ID | untreated |
| NO: | NO: | at 20 nM) |
| 193 | 385 | 69.63 |
| 194 | 386 | 77.15 |
| 195 | 387 | 86.61 |
| 196 | 388 | 86.93 |
| 197 | 389 | 83.34 |
| 198 | 390 | 76.14 |
| 199 | 391 | 80.15 |
| 200 | 392 | 72.23 |
| 201 | 393 | 85.37 |
| 202 | 394 | 91.28 |
| 203 | 395 | 70.24 |
| 204 | 396 | 78.53 |
| 205 | 397 | 76.26 |
| 206 | 398 | 77.72 |
| 207 | 399 | 75.12 |
| 208 | 400 | 83.97 |
| 209 | 401 | 79.62 |
| 210 | 402 | 76.45 |
| 211 | 403 | 69.28 |
| 212 | 404 | 68.75 |
| 213 | 405 | 68.75 |
| 214 | 406 | 94.23 |
| 215 | 407 | 95.35 |
| 216 | 408 | 79.64 |
| 217 | 409 | 89.69 |
| 218 | 410 | 94.79 |
| 219 | 411 | 81.76 |
| 220 | 412 | 71.71 |
| 221 | 413 | 85.23 |
| 222 | 414 | 87.67 |
| 223 | 415 | 71.41 |
| 224 | 416 | 58.05 |
| 225 | 417 | 52.44 |
| 226 | 418 | 90.83 |
| 227 | 419 | 79.27 |
| 228 | 420 | 84.94 |
| 229 | 421 | 59.79 |
| 230 | 422 | 67.05 |
| 231 | 423 | 84.80 |
| 232 | 424 | 84.69 |
| 233 | 425 | 79.85 |
| 234 | 426 | 87.66 |
| 235 | 427 | 94.58 |
| 236 | 428 | 60.04 |
| 237 | 429 | 70.44 |
| 238 | 430 | 89.00 |
| 239 | 431 | 68.03 |
| 240 | 432 | 97.43 |
| 241 | 433 | 97.68 |
| 242 | 434 | 79.78 |
| 243 | 435 | 92.24 |
| 244 | 436 | 81.56 |
| 245 | 437 | 74.92 |
| 246 | 438 | 75.73 |
| 247 | 439 | 75.37 |
| 248 | 440 | 80.74 |
| 249 | 441 | 92.38 |
| 250 | 442 | 81.68 |
| 251 | 443 | 57.88 |
| 252 | 444 | 88.61 |
| 253 | 445 | 51.39 |
| 254 | 446 | 87.28 |
| 255 | 447 | 77.63 |
| 256 | 448 | 91.15 |
| 257 | 449 | 92.04 |
| 258 | 450 | 60.58 |
| 259 | 451 | 64.59 |
| 260 | 452 | 87.53 |
| 261 | 453 | 65.98 |
| 262 | 454 | 94.82 |
| 263 | 455 | 77.65 |
| 264 | 456 | 74.48 |
| 265 | 457 | 56.02 |
| 266 | 458 | 87.59 |
| 267 | 459 | 95.52 |
| 268 | 460 | 86.64 |
| 269 | 461 | 96.34 |
| 270 | 462 | 77.63 |
| 271 | 463 | 72.27 |
| 272 | 464 | 82.56 |
| 273 | 465 | 57.71 |
| 274 | 466 | 92.02 |
| 275 | 467 | 71.19 |
| 276 | 468 | 81.90 |
| 277 | 469 | 79.61 |
| 278 | 470 | 90.88 |
| 279 | 471 | 74.17 |
| 280 | 472 | 73.91 |
| 281 | 473 | 91.20 |
| 282 | 474 | 81.92 |
| 283 | 475 | 83.81 |
| 284 | 476 | 94.81 |
| 285 | 477 | 86.85 |
| 286 | 478 | 89.57 |
| 287 | 479 | 90.60 |
| 288 | 480 | 93.38 |
| 289 | 481 | 68.19 |
| 290 | 482 | 71.75 |
| 291 | 483 | 61.61 |
| 292 | 484 | 65.85 |
| 293 | 485 | 59.82 |
| 294 | 486 | 92.71 |
| 295 | 487 | 102.50 |
| 296 | 488 | 99.64 |
| 297 | 489 | 91.28 |
| 298 | 490 | 76.47 |
| 299 | 491 | 68.83 |
| 300 | 492 | 90.72 |
| 301 | 493 | 65.64 |
| 302 | 494 | 49.76 |
| 303 | 495 | 83.85 |
| 304 | 496 | 73.80 |
| 305 | 497 | 84.33 |
| 311 | 503 | 84.57 |
| 312 | 504 | 73.46 |
| 313 | 505 | 82.60 |
| 314 | 506 | 70.05 |
| 315 | 507 | 65.74 |
| 316 | 508 | 71.20 |
| 317 | 509 | 77.12 |
| 318 | 510 | 78.76 |
| 319 | 511 | 81.97 |
| 320 | 512 | 103.57 |
| 321 | 513 | 107.03 |
| 322 | 514 | 101.64 |
| 323 | 515 | 96.73 |
| 324 | 516 | 105.55 |
| 325 | 517 | 74.82 |
| 326 | 518 | 88.77 |
| 327 | 519 | 70.21 |
| 328 | 520 | 81.19 |
| 329 | 521 | 60.24 |
| 330 | 522 | 81.62 |
| 331 | 523 | 89.41 |
| 332 | 524 | 98.67 |
| 333 | 525 | 107.27 |
| 334 | 526 | 88.03 |
| 335 | 527 | 73.04 |
| 336 | 528 | 65.16 |
| 337 | 529 | 62.91 |
| 338 | 530 | 74.95 |
| 339 | 531 | 77.75 |
| 340 | 532 | 64.53 |
| 341 | 533 | 80.50 |
| 342 | 534 | 82.81 |
| 343 | 535 | 105.71 |
| 344 | 536 | 77.78 |
| 345 | 537 | 77.38 |
| 346 | 538 | 70.18 |
| 347 | 539 | 76.40 |
| 348 | 540 | 98.60 |
| 349 | 541 | 78.42 |
| 350 | 542 | 74.37 |
| 351 | 543 | 62.86 |
| 352 | 544 | 68.25 |
| 353 | 545 | 57.73 |
| 354 | 546 | 70.46 |
| 355 | 547 | 90.61 |
| 356 | 548 | 92.04 |
| 357 | 549 | 105.13 |
| 358 | 550 | 100.91 |
| 359 | 551 | 85.32 |
| 360 | 552 | 103.66 |
| 361 | 553 | 93.76 |
| 362 | 554 | 89.06 |
| 363 | 555 | 76.58 |
| 364 | 556 | 77.26 |
| 365 | 557 | 83.69 |
| 366 | 558 | 97.41 |
| 367 | 559 | 94.55 |
| 368 | 560 | 89.86 |
| 369 | 561 | 103.68 |
| 370 | 562 | 90.59 |
| 371 | 563 | 93.80 |
| 372 | 564 | 89.39 |
| 373 | 565 | 75.20 |
| 374 | 566 | 77.15 |
| 375 | 567 | 92.61 |
| 376 | 568 | 93.87 |
| 377 | 569 | 91.59 |
| 378 | 570 | 111.67 |
| 379 | 571 | 105.64 |
| 380 | 572 | 111.48 |
| 381 | 573 | 106.51 |
| 382 | 574 | 112.30 |
| 383 | 575 | 95.68 |
| 384 | 576 | 98.33 |

TABLE 4
ATXN3 knockdown with siRNA molecules
of the disclosure at 0.2 nM

| | | Residual |
|---|---|---|
| | | mRNA |
| | | expression |
| Sense | Antisense | (% of |
| SEQ ID | SEQ ID | untreated |
| NO: | NO: | at 0.2 nM) |
| 193 | 385 | 103.84 |
| 194 | 386 | 105.70 |
| 195 | 387 | 106.94 |
| 196 | 388 | 106.22 |
| 197 | 389 | 104.84 |
| 198 | 390 | 105.74 |
| 199 | 391 | 108.71 |
| 200 | 392 | 104.79 |
| 201 | 393 | 105.01 |
| 202 | 394 | 105.09 |
| 203 | 395 | 103.41 |
| 204 | 396 | 105.07 |
| 205 | 397 | 113.55 |
| 206 | 398 | 106.38 |
| 207 | 399 | 110.71 |
| 208 | 400 | 110.45 |
| 209 | 401 | 107.70 |
| 210 | 402 | 103.16 |
| 211 | 403 | 101.16 |
| 212 | 404 | 105.93 |
| 213 | 405 | 108.13 |
| 214 | 406 | 105.40 |
| 215 | 407 | 100.72 |
| 216 | 408 | 105.55 |
| 217 | 409 | 112.76 |
| 218 | 410 | 111.10 |
| 219 | 411 | 110.94 |
| 220 | 412 | 113.03 |
| 221 | 413 | 117.03 |
| 222 | 414 | 109.26 |
| 223 | 415 | 107.50 |
| 224 | 416 | 107.44 |
| 225 | 417 | 106.93 |
| 226 | 418 | 106.13 |
| 227 | 419 | 99.39 |
| 228 | 420 | 108.22 |
| 229 | 421 | 104.53 |
| 230 | 422 | 110.00 |
| 231 | 423 | 108.61 |
| 232 | 424 | 109.01 |
| 233 | 425 | 111.11 |
| 234 | 426 | 107.56 |
| 235 | 427 | 105.71 |
| 236 | 428 | 103.79 |
| 237 | 429 | 105.81 |
| 238 | 430 | 113.19 |
| 239 | 431 | 102.95 |
| 240 | 432 | 111.01 |
| 241 | 433 | 103.39 |
| 242 | 434 | 111.22 |
| 243 | 435 | 109.74 |
| 244 | 436 | 110.26 |
| 245 | 437 | 107.95 |
| 246 | 438 | 104.93 |
| 247 | 439 | 103.68 |
| 248 | 440 | 111.10 |
| 249 | 441 | 110.12 |
| 250 | 442 | 105.67 |
| 251 | 443 | 101.19 |
| 252 | 444 | 110.98 |
| 253 | 445 | 100.21 |
| 254 | 446 | 112.12 |
| 255 | 447 | 108.78 |
| 256 | 448 | 107.77 |
| 257 | 449 | 105.95 |
| 258 | 450 | 104.29 |
| 259 | 451 | 107.40 |
| 260 | 452 | 107.59 |
| 261 | 453 | 106.70 |
| 262 | 454 | 108.60 |
| 263 | 455 | 104.41 |
| 264 | 456 | 107.14 |
| 265 | 457 | 100.18 |
| 266 | 458 | 107.87 |
| 267 | 459 | 111.23 |
| 268 | 460 | 106.75 |
| 269 | 461 | 107.57 |
| 270 | 462 | 104.29 |
| 271 | 463 | 103.98 |
| 272 | 464 | 105.15 |
| 273 | 465 | 102.89 |
| 274 | 466 | 102.03 |
| 275 | 467 | 98.32 |
| 276 | 468 | 99.12 |
| 277 | 469 | 93.14 |
| 278 | 470 | 93.50 |
| 279 | 471 | 94.59 |
| 280 | 472 | 91.65 |
| 281 | 473 | 97.76 |
| 282 | 474 | 97.11 |
| 283 | 475 | 99.87 |
| 284 | 476 | 97.88 |
| 285 | 477 | 98.96 |
| 286 | 478 | 98.34 |
| 287 | 479 | 99.61 |
| 288 | 480 | 98.71 |
| 289 | 481 | 103.26 |
| 290 | 482 | 101.76 |
| 291 | 483 | 100.62 |
| 292 | 484 | 107.34 |
| 293 | 485 | 99.43 |
| 294 | 486 | 102.05 |
| 295 | 487 | 104.83 |
| 296 | 488 | 107.41 |
| 297 | 489 | 101.63 |
| 298 | 490 | 103.62 |
| 299 | 491 | 106.54 |
| 300 | 492 | 110.07 |
| 301 | 493 | 109.40 |
| 302 | 494 | 99.20 |
| 303 | 495 | 102.67 |
| 304 | 496 | 102.24 |
| 305 | 497 | 101.66 |
| 306 | 498 | 105.28 |
| 307 | 499 | 104.81 |
| 308 | 500 | 107.66 |
| 309 | 501 | 105.07 |
| 310 | 502 | 109.33 |
| 311 | 503 | 111.66 |
| 312 | 504 | 109.42 |
| 313 | 505 | 100.93 |
| 314 | 506 | 102.68 |
| 315 | 507 | 102.53 |
| 316 | 508 | 103.85 |
| 317 | 509 | 105.36 |
| 318 | 510 | 102.36 |
| 319 | 511 | 109.72 |
| 320 | 512 | 105.53 |
| 321 | 513 | 106.91 |
| 322 | 514 | 112.68 |
| 323 | 515 | 114.51 |
| 324 | 516 | 117.23 |
| 325 | 517 | 103.14 |
| 326 | 518 | 110.06 |
| 327 | 519 | 104.26 |
| 328 | 520 | 106.41 |
| 329 | 521 | 105.52 |
| 330 | 522 | 94.90 |
| 331 | 523 | 106.04 |
| 332 | 524 | 101.69 |
| 333 | 525 | 110.09 |
| 334 | 526 | 115.48 |
| 335 | 527 | 112.86 |
| 336 | 528 | 115.05 |
| 337 | 529 | 99.29 |
| 338 | 530 | 104.00 |
| 339 | 531 | 103.53 |
| 340 | 532 | 103.51 |
| 341 | 533 | 107.23 |
| 342 | 534 | 101.92 |
| 343 | 535 | 102.18 |
| 344 | 536 | 108.40 |
| 345 | 537 | 106.13 |
| 346 | 538 | 107.91 |
| 347 | 539 | 112.88 |
| 348 | 540 | 114.70 |
| 349 | 541 | 100.47 |
| 350 | 542 | 104.24 |
| 351 | 543 | 102.46 |
| 352 | 544 | 101.61 |
| 353 | 545 | 104.12 |
| 354 | 546 | 103.10 |
| 355 | 547 | 100.01 |
| 356 | 548 | 110.14 |
| 357 | 549 | 106.10 |
| 358 | 550 | 107.31 |
| 359 | 551 | 110.42 |
| 360 | 552 | 111.95 |
| 361 | 553 | 102.61 |
| 362 | 554 | 102.17 |
| 363 | 555 | 101.99 |
| 364 | 556 | 104.13 |
| 365 | 557 | 98.19 |
| 366 | 558 | 100.47 |
| 367 | 559 | 99.88 |
| 368 | 560 | 101.10 |
| 369 | 561 | 107.17 |
| 370 | 562 | 104.09 |
| 371 | 563 | 106.57 |
| 372 | 564 | 107.45 |
| 373 | 565 | 97.47 |
| 374 | 566 | 94.87 |
| 375 | 567 | 103.13 |
| 376 | 568 | 94.47 |
| 377 | 569 | 96.47 |
| 378 | 570 | 100.53 |
| 379 | 571 | 95.16 |
| 380 | 572 | 101.88 |
| 381 | 573 | 104.57 |
| 382 | 574 | 102.81 |
| 383 | 575 | 104.71 |
| 384 | 576 | 101.34 |

### Example 2. Generating ATXN3-Targeting siRNA Molecules

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the ATXN3 mRNA target sequence, are shown in Table 1, above. It is appreciated that one of skill in the art could anneal the antisense (AS) strand to the corresponding sense (5) strand to yield a ds-siRNA molecule. Alternatively, one of skill in the art could derive a ss-siRNA molecule using antisense strand only.

### Example 3. Optimizing ATXN3-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 4. Preparation and Administrating ATXN3-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject that is tolerated by the siRNA compositions of the disclosure. Non-limiting examples of siRNA injections into the CNS include intrathecally, intracerebroventricularly, intrastriatally, intraparenchymally, or intra-cisterna magna injection by catheterization. Examples of systemic administration include intravenous, intramuscular, and subcutaneous injection. A physician having ordinary skill in the art can readily determine an effective route of administration.

### Example 5. Methods for the Treatment of a Spinocerebellar Ataxia Using ATXN3-Targeting siRNA Molecules

A subject in need of treatment of a spinocerebellar ataxia, such as spinocerebellar ataxia type 3 (SCA3), is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of ATXN3 mRNA or suitable biomarker or a reduction of SCA-associated phenotypes) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, intrastriatally, intraparenchymally, intravenously, intramuscularly, subcutaneously, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally, intraparenchymally, intravenously, intramuscularly, subcutaneously, or by intra-cisterna magna injection via catheterization) and condition at a rate tolerable to the patient until the subject has reached a maximum tolerated dose, or until symptoms are ameliorated satisfactorily.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within an ataxin-3 (ATXN3) mRNA transcript having the nucleic acid sequence of any one of SEQ ID NOs: 1-192.

2. The siRNA molecule of claim 1, wherein the antisense strand has at least 70% complementarity to a region of 19, 20, 21, or more contiguous nucleobases within the ATXN3 mRNA transcript having the nucleic acid sequence of any one of SEQ ID NOs: 1-192, optionally wherein the antisense strand has at least 70% complementarity to the ATXN3 mRNA transcript having the nucleic acid sequence of any one of SEQ ID NOs: 1-192.

3. The siRNA molecule of claim 2, wherein the antisense strand has at least 75% complementarity to a region of 21 contiguous nucleobases within the ATXN3 mRNA transcript having the nucleic acid sequence of any one of SEQ ID NOs: 1-192, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the ATXN3 mRNA transcript having the nucleic acid sequence of any one of SEQ ID Nos: 1-192.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the ATXN3 RNA transcript having the nucleic acid sequence of any one of SEQ ID NOs: 1-192.

5. The siRNA molecule of claim 4, wherein the antisense strand comprises from 10 to 30 contiguous nucleotides that are fully complementary to a contiguous polynucleotide segment of equal length within the region of the ATXN3 RNA transcript having the nucleic acid sequence of any one of SEQ ID NOs: 1-192.

6. The siRNA molecule of claim 5, wherein the antisense strand comprises from 12 to 30 contiguous nucleotides that are fully complementary to a contiguous polynucleotide segment of equal length within the region of the ATXN3 RNA transcript having the nucleic acid sequence of any one of SEQ ID NOs: 1-192.

7. The siRNA molecule of claim 6, wherein the antisense strand comprises from 15 to 30 contiguous nucleotides that are fully complementary to a contiguous polynucleotide segment of equal length within the region of the ATXN3 RNA transcript having the nucleic acid sequence of any one of SEQ ID NOs: 1-192.

8. The siRNA molecule of claim 7, wherein the antisense strand comprises from 18 to 30 contiguous nucleotides that are fully complementary to a contiguous polynucleotide segment of equal length within the region of the ATXN3 RNA transcript having the nucleic acid sequence of any one of SEQ ID NOs: 1-192.

9. The siRNA molecule of claim 8, wherein the antisense strand comprises from 18 to 25 contiguous nucleotides that are fully complementary to a contiguous polynucleotide segment of equal length within the region of the ATXN3 RNA transcript having the nucleic acid sequence of any one of SEQ ID NOs: 1-192.

10. The siRNA molecule of any one of claim 9, wherein the antisense strand comprises from 18 to 21 contiguous nucleotides that are fully complementary to a contiguous polynucleotide segment of equal length within the region of the ATXN3 RNA transcript having the nucleic acid sequence of any one of SEQ ID NOs: 1-192.

11. The siRNA molecule of claim 10, wherein the antisense strand comprises 21 contiguous nucleotides that are fully complementary to a contiguous polynucleotide segment of equal length within the region of the ATXN3 RNA transcript having the nucleic acid sequence of any one of SEQ ID NOs: 1-192.

12. The siRNA molecule of any one of claims 1-11, wherein the antisense strand comprises 9 or fewer nucleotide mismatches relative to a region of 21 contiguous nucleobases of the ATXN3 RNA transcript having the nucleic acid sequence of any one of SEQ ID NOs: 1-192, optionally wherein the antisense strand comprises 8 or fewer, 7 or fewer, 6 or fewer, 5 or fewer, 4 or fewer, 3 or fewer, 2 or fewer, or only 1 mismatch relative to the region of the ATXN3 RNA transcript having the nucleic acid sequence of any one of SEQ ID NOs: 1-192.

13. The siRNA molecule of any one of claims 1-12, wherein the antisense strand has a nucleic acid sequence that is at least 85% identical to the nucleic acid sequence of any one of SEQ ID NOs: 385-576.

14. The siRNA molecule of claim 13, wherein the antisense strand has a nucleic acid sequence that is at least 90% identical to the nucleic acid sequence of any one of SEQ ID NOs: 385-576.

15. The siRNA molecule of claim 14, wherein the antisense strand has a nucleic acid sequence that is at least 95% identical to the nucleic acid sequence of SEQ ID NOs: 385-576, optionally wherein the antisense strand has a nucleic acid sequence that is at least 96%, 97%, 98%, or 99% identical to the nucleic acid sequence of any one of SEQ ID NOs: 385-576.

16. The siRNA molecule of claim 15, wherein the antisense strand has the nucleic acid sequence of any one of SEQ ID NOs: 385-576.

17. The siRNA molecule of any one of claims 1-16, wherein the sense strand has a nucleic acid sequence that is at least 85% identical to the nucleic acid sequence of any one of SEQ ID NOs: 193-384.

18. The siRNA molecule of claim 17, wherein the sense strand has a nucleic acid sequence that is at least 90% identical to the nucleic acid sequence of any one of SEQ ID NOs: 193-384.

19. The siRNA molecule of claim 18, wherein the sense strand has a nucleic acid sequence that is at least 95% identical to the nucleic acid sequence of SEQ ID NOs: 193-384, optionally wherein the sense strand has a nucleic acid sequence that is at least 96%, 97%, 98%, or 99% identical to the nucleic acid sequence of any one of SEQ ID NOs: 193-384.

20. The siRNA molecule of claim 19, wherein the sense strand has the nucleic acid sequence of any one of SEQ ID NOs: 193-384.

21. The siRNA molecule of any one of claims 1-20, wherein the antisense strand comprises a structure represented by Formula I, wherein Formula I is, in the 5′-to-3′ direction: 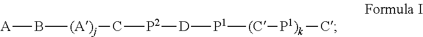
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

22. The siRNA molecule of claim 21, wherein the antisense strand comprises a structure represented by Formula A1, wherein Formula A1 is, in the 5′-to-3′ direction: 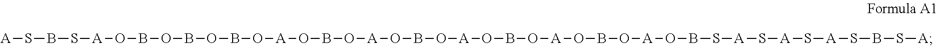
wherein A represents a 2′-O-Me ribonucleoside, B represents a 2′-F ribonucleoside, O represents a phosphodiester internucleoside linkage, and S represents a phosphorothioate internucleoside linkage.

23. The siRNA molecule of any one of claims 1-20, wherein the antisense strand comprises a structure represented by Formula II, wherein Formula II is, in the 5′-to-3′ direction: 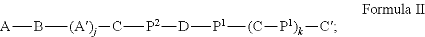
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

24. The siRNA molecule of claim 23, wherein the antisense strand comprises a structure represented by Formula A2, wherein Formula A2 is, in the 5′-to-3′ direction: 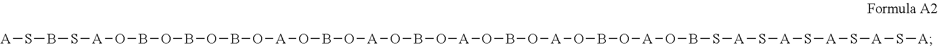
wherein A represents a 2′-O-Me ribonucleoside, B represents a 2′-F ribonucleoside, O represents a phosphodiester internucleoside linkage, and S represents a phosphorothioate internucleoside linkage.

25. The siRNA molecule of any one of claims 1-24, wherein the sense strand comprises a structure represented by Formula III, wherein Formula III is, in the 5′-to-3′ direction: 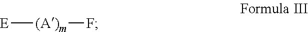
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

26. The siRNA molecule of claim 25, wherein the sense strand comprises a structure represented by Formula S1, wherein Formula S1 is, in the 5′-to-3′ direction: 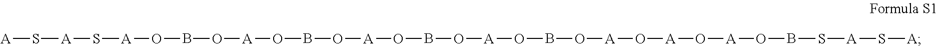
wherein A represents a 2′-O-Me ribonucleoside, B represents a 2′-F ribonucleoside, O represents a phosphodiester internucleoside linkage, and S represents a phosphorothioate internucleoside linkage.

27. The siRNA molecule of claim 25, wherein the sense strand comprises a structure represented by Formula S2, wherein Formula S2 is, in the 5′-to-3′ direction: 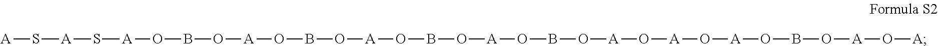
wherein A represents a 2′-O-Me ribonucleoside, B represents a 2′-F ribonucleoside, O represents a phosphodiester internucleoside linkage, and S represents a phosphorothioate internucleoside linkage.

28. The siRNA molecule of claim 25, wherein the sense strand comprises a structure represented by Formula S3, wherein Formula S3 is, in the 5′-to-3′ direction: 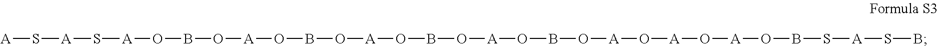
wherein A represents a 2′-O-Me ribonucleoside, B represents a 2′-F ribonucleoside, O represents a phosphodiester internucleoside linkage, and S represents a phosphorothioate internucleoside linkage.

29. The siRNA molecule of claim 25, wherein the sense strand comprises a structure represented by Formula S4, wherein Formula S4 is, in the 5′-to-3′ direction: 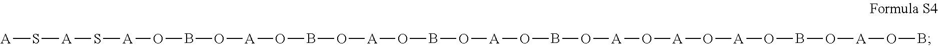
wherein A represents a 2′-O-Me ribonucleoside, B represents a 2′-F ribonucleoside, O represents a phosphodiester internucleoside linkage, and S represents a phosphorothioate internucleoside linkage.

30. The siRNA molecule of any one of claims 1-20 and 25-29, wherein the antisense strand comprises a structure represented by Formula IV, wherein Formula IV is, in the 5′-to-3′ direction: 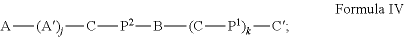
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

31. The siRNA molecule of claim 30, wherein the antisense strand comprises a structure represented by Formula A3, wherein Formula A3 is, in the 5′-to-3′ direction: 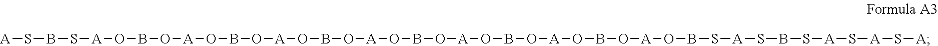
wherein A represents a 2′-O-Me ribonucleoside, B represents a 2′-F ribonucleoside, O represents a phosphodiester internucleoside linkage, and S represents a phosphorothioate internucleoside linkage.

32. The siRNA molecule of any one of claims 1-24, 30, and 31, wherein the sense strand comprises a structure represented by Formula V, wherein Formula V is, in the 5′-to-3′ direction: 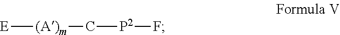
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

33. The siRNA molecule of claim 32, wherein the sense strand comprises a structure represented by Formula S5, wherein Formula S5 is, in the 5′-to-3′ direction: 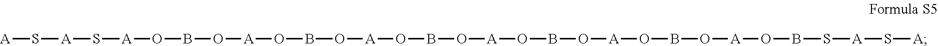
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of claim 32, wherein the sense strand comprises a structure represented by Formula S6, wherein Formula S6 is, in the 5′-to-3′ direction: 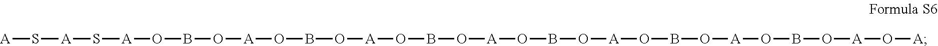
wherein A represents a 2′-O-Me ribonucleoside, B represents a 2′-F ribonucleoside, O represents a phosphodiester internucleoside linkage, and S represents a phosphorothioate internucleoside linkage.

35. The siRNA molecule of claim 32, wherein the sense strand comprises a structure represented by Formula S7, wherein Formula S7 is, in the 5′-to-3′ direction: 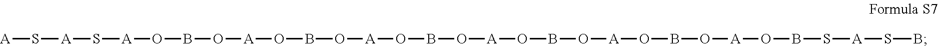
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of claim 32, wherein the sense strand comprises a structure represented by Formula S8, wherein Formula S8 is, in the 5′-to-3′ direction: 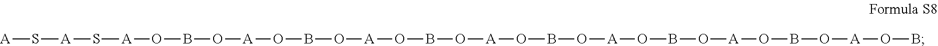
wherein A represents a 2′-O-Me ribonucleoside, B represents a 2′-F ribonucleoside, O represents a phosphodiester internucleoside linkage, and S represents a phosphorothioate internucleoside linkage.

37. The siRNA molecule of any one of claims 1-20, 25-29 and 32-36, wherein the antisense strand comprises a structure represented by Formula VI, wherein Formula VI is, in the 5′-to-3′ direction: 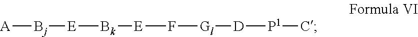
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

38. The siRNA molecule of claim 37, wherein the antisense strand comprises a structure represented by Formula A4, wherein Formula A4 is, in the 5′-to-3′ direction: 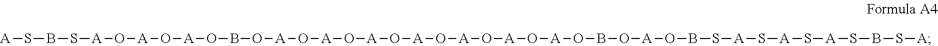
wherein A represents a 2′-O-Me ribonucleoside, B represents a 2′-F ribonucleoside, O represents a phosphodiester internucleoside linkage, and S represents a phosphorothioate internucleoside linkage.

39. The siRNA molecule of any one of claims 1-24, 30, 31, 37, and 38, wherein the sense strand comprises a structure represented by Formula VII, wherein Formula VII is, in the 5′-to-3′ direction: 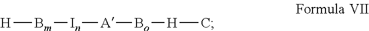
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

40. The siRNA molecule of claim 39, wherein the sense strand comprises a structure represented by Formula S9, wherein Formula S9 is, in the 5′-to-3′ direction: 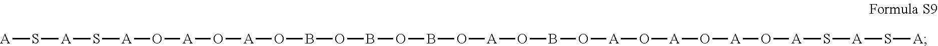
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of any one of claims 1-40, wherein the antisense strand further comprises a 5′ phosphorus stabilizing moiety at the 5′ end of the antisense strand.

42. The siRNA molecule of any one of claims 1-41, wherein the sense strand further comprises a 5′ phosphorus stabilizing moiety at the 5′ end of the sense strand.

43. The siRNA molecule of claim 41 or 42, wherein each 5′ phosphorus stabilizing moiety is, independently, represented by any one of Formulas IX-XVI: 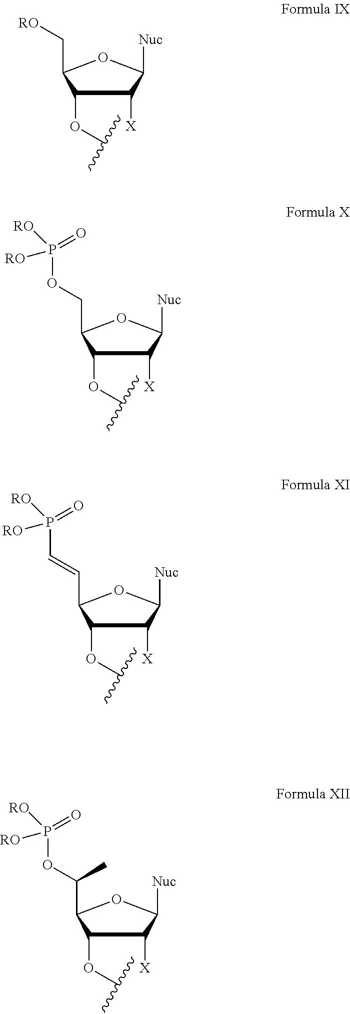 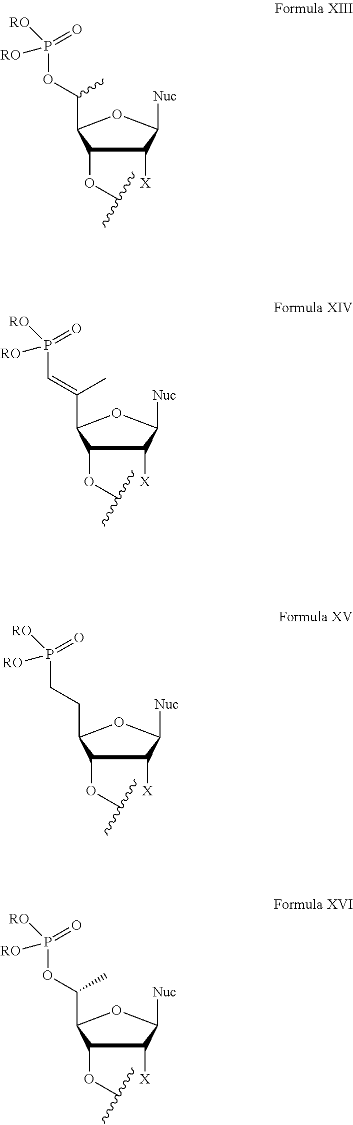
wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation, or hydrogen.

44. The siRNA molecule of claim 43, wherein the nucleobase is an adenine, uracil, guanine, thymine, or cytosine.

45. The siRNA molecule of any one of claims 41-44, wherein the 5′ phosphorus stabilizing moiety is (E)-vinylphosphonate represented by Formula XI.

46. The siRNA molecule of any one of claims 1-45, wherein the siRNA molecule further comprises a hydrophobic moiety at the 5′ or the 3′ end of the siRNA molecule.

47. The siRNA molecule of claim 46, wherein the hydrophobic moiety is selected from a group consisting of cholesterol, vitamin D, or tocopherol.

48. The siRNA molecule of any one of claims 1-47, wherein the length of the sense strand is between 10 and 30 nucleotides.

49. The siRNA molecule of claim 48, wherein the length of the sense strand is between 10 and 25 nucleotides.

50. The siRNA molecule of claim 49, wherein the length of the sense strand is between 12 and 25 nucleotides.

51. The siRNA molecule of claim 50, wherein the length of the sense strand is between 12 and 20 nucleotides.

52. The siRNA molecule of claim 51, wherein the length of the sense strand is between 12 and 19 nucleotides.

53. The siRNA molecule of claim 52, wherein the length of the sense strand is 15 nucleotides.

54. The siRNA molecule of claim 52, wherein the length of the sense strand is 16 nucleotides.

55. The siRNA molecule of claim 52, wherein the length of the sense strand is 18 nucleotides.

56. The siRNA molecule of any one of claims 1-55, wherein the length of the antisense strand is between 10 and 30 nucleotides.

57. The siRNA molecule of claim 56, wherein the length of the antisense strand is between 12 and 30 nucleotides.

58. The siRNA molecule of claim 57, wherein the length of the antisense strand is between 15 and 30 nucleotides.

59. The siRNA molecule of claim 58, wherein the length of the antisense strand is between 18 and 30 nucleotides.

60. The siRNA molecule of claim 59, wherein the length of the antisense strand is between 18 and 25 nucleotides.

61. The siRNA molecule of claim 60, wherein the length of the antisense strand is between 18 and 21 nucleotides.

62. The siRNA molecule of claim 61, wherein the length of the antisense strand is 18 nucleotides.

63. The siRNA molecule of claim 61, wherein the length of the antisense strand is 20 nucleotides.

64. The siRNA molecule of claim 61, wherein the length of the antisense strand is 21 nucleotides.

65. The siRNA molecule of any one of claims 1-64, wherein the siRNA molecule is a branched siRNA molecule.

66. The siRNA molecule of claim 65, wherein the branched siRNA molecule is di-branched, tri-branched, or tetra-branched.

67. The siRNA molecule of claim 66, wherein the siRNA molecule is a di-branched siRNA molecule, optionally wherein the di-branched siRNA molecule is represented by any one of Formulas XVII-XIX: 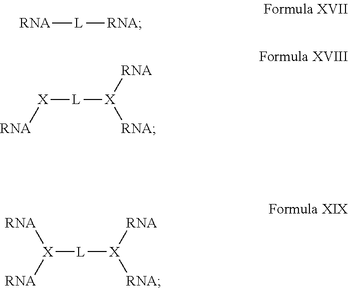
wherein each RNA is, independently, an siRNA molecule, L is a linker, and each X, independently, represents a branch point moiety.

68. The siRNA molecule of claim 66, wherein the siRNA molecule is a tri-branched siRNA molecule, optionally wherein the tri-branched siRNA molecule is represented by any one of Formulas XX-XXIII: 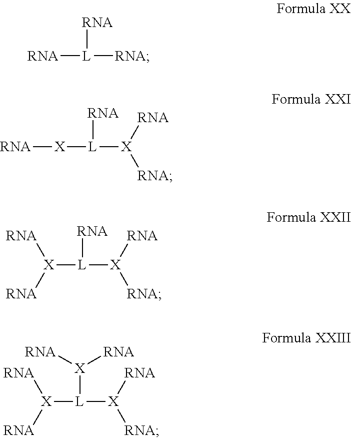
wherein each RNA is, independently, an siRNA molecule, L is a linker, and each X, independently, represents a branch point moiety.

69. The siRNA molecule of claim 66, wherein the siRNA molecule is a tetra-branched siRNA molecule, optionally wherein the tetra-branched siRNA molecule is represented by any one of Formulas XXIV-XXVIII: 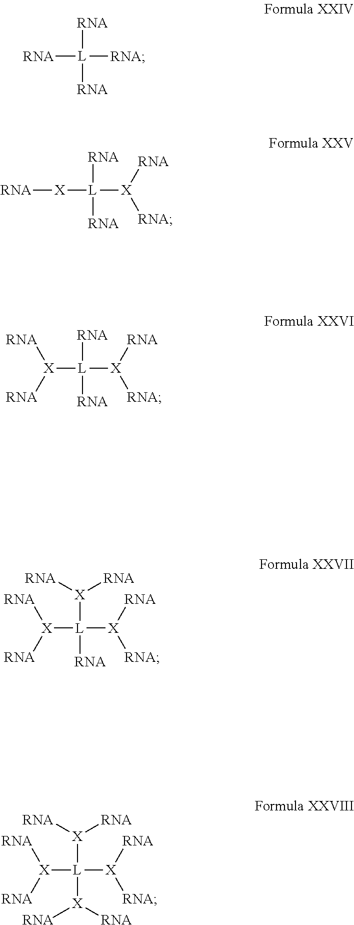
wherein each RNA is, independently, an siRNA molecule, L is a linker, and each X, independently, represents a branch point moiety.

70. The siRNA molecule of any one of claims 67-69, wherein the linker is selected from a group consisting of one or more contiguous subunits of an ethylene glycol, alkyl, carbohydrate, block copolymer, peptide, RNA, and DNA.

71. The siRNA molecule of claim 70, wherein the one or more contiguous subunits is 2 to 20 contiguous subunits.

72. A pharmaceutical composition comprising the siRNA molecule of any one of claims 1-71 and a pharmaceutically acceptable excipient, carrier, or diluent.

73. A method of delivering an siRNA molecule to a subject diagnosed as having a spinocerebellar ataxia, the method comprising administering a therapeutically effective amount of the siRNA molecule of any one of claims 1-71 or the pharmaceutical composition of claim 72 to the subject.

74. A method of treating a spinocerebellar ataxia in a subject in need thereof, the method comprising administering a therapeutically effective amount of the siRNA molecule of any one of claims 1-71 or the pharmaceutical composition of claim 72 to the subject.

75. The method of claim 73 or 74, wherein the spinocerebellar ataxia is spinocerebellar ataxia type 3 (SCA3).

76. A method of reducing ATXN3 expression in a subject in need thereof, the method comprising administering a therapeutically effective amount of the siRNA molecule of any one of claims 1-71 or the pharmaceutical composition of claim 72 to the subject.

77. The method of any one of claims 73-76, wherein the siRNA molecule or the pharmaceutical composition is administered to the subject by way of intracerebroventricular, intrastriatal, intraparenchymal, or intrathecal injection.

78. The method of any one of claims 73-76, wherein the siRNA molecule or the pharmaceutical composition is administered to the subject by way of intravenous, intramuscular, or subcutaneous injection.

79. The method of any one of claims 73-78, wherein the subject is a human.

80. A kit comprising the siRNA molecule of any one of claims 1-71, or the pharmaceutical composition of claim 72, and a package insert, wherein the package insert instructs a user of the kit to perform the method of any one of claims 73-79.


SEQUENCE LISTING (576 sequences)

SEQ ID NO: 1 - RNA, 21 nt, Homo sapiens
  source 1..21: mol_type = mRNA
aaacaagaaggctcactttgt

SEQ ID NO: 2 - RNA, 21 nt, Homo sapiens
  source 1..21: mol_type = mRNA
aacaagaaggctcactttgtg

SEQ ID NO: 3 - RNA, 21 nt, Homo sapiens
  source 1..21: mol_type = mRNA
acaagaaggctcactttgtgc

SEQ ID NO: 4 - RNA, 21 nt, Homo sapiens
  source 1..21: mol_type = mRNA
caagaaggctcactttgtgct

SEQ ID NO: 5 - RNA, 21 nt, Homo sapiens
  source 1..21: mol_type = mRNA
agaaggctcactttgtgctca

SEQ ID NO: 6 - RNA, 21 nt, Homo sapiens
  source 1..21: mol_type = mRNA
cactttgtgctcaacattgcc

SEQ ID NO: 7 - RNA, 21 nt, Homo sapiens
  source 1..21: mol_type = mRNA
gctcaacattgcctgaataac

SEQ ID NO: 8 - RNA, 21 nt, Homo sapiens
  source 1..21: mol_type = mRNA
taacttattgcaaggagaata

SEQ ID NO: 9 - RNA, 21 nt, Homo sapiens
  source 1..21: mol_type = mRNA
ttttagccctgtggaattatc

SEQ ID NO: 10 - RNA, 21 nt, Homo sapiens
  source 1..21: mol_type = mRNA
tttagccctgtggaattatcc

SEQ ID NO: 11 - RNA, 21 nt, Homo sapiens
  source 1..21: mol_type = mRNA
tagccctgtggaattatcctc

SEQ ID NO: 12 - RNA, 21 nt, Homo sapiens
  source 1..21: mol_type = mRNA
gtggaattatcctcaattgca

SEQ ID NO: 13 - RNA, 21 nt, Homo sapiens
  source 1..21: mol_type = mRNA
ggaattatcctcaattgcaca

SEQ ID NO: 14 - RNA, 21 nt, Homo sapiens
  source 1..21: mol_type = mRNA
gatgaggaggagaggatgaga

SEQ ID NO: 15 - RNA, 21 nt, Homo sapiens
  source 1..21: mol_type = mRNA
atggcagaaggaggagttact

SEQ ID NO: 16 - RNA, 21 nt, Homo sapiens
  source 1..21: mol_type = mRNA
gcagaaggaggagttactagt

SEQ ID NO: 17 - RNA, 21 nt, Homo sapiens
  source 1..21: mol_type = mRNA
gaaggaggagttactagtgaa

SEQ ID NO: 18 - RNA, 21 nt, Homo sapiens
  source 1..21: mol_type = mRNA
gagttactagtgaagattatc

SEQ ID NO: 19 - RNA, 21 nt, Homo sapiens
  source 1..21: mol_type = mRNA
agttactagtgaagattatcg

SEQ ID NO: 20 - RNA, 21 nt, Homo sapiens
  source 1..21: mol_type = mRNA
ttactagtgaagattatcgca

SEQ ID NO: 21 - RNA, 21 nt, Homo sapiens
  source 1..21: mol_type = mRNA
ttatcgcacgtttttacagca

SEQ ID NO: 22 - RNA, 21 nt, Homo sapiens
  source 1..21: mol_type = mRNA
cgtttttacagcagccttctg

SEQ ID NO: 23 - RNA, 21 nt, Homo sapiens
  source 1..21: mol_type = mRNA
gtttttacagcagccttctgg

SEQ ID NO: 24 - RNA, 21 nt, Homo sapiens
  source 1..21: mol_type = mRNA
tttttacagcagccttctgga

SEQ ID NO: 25 - RNA, 21 nt, Homo sapiens
  source 1..21: mol_type = mRNA
acagcagccttctggaaatat

SEQ ID NO: 26 - RNA, 21 nt, Homo sapiens
  source 1..21: mol_type = mRNA
gcagccttctggaaatatgga

SEQ ID NO: 27 - RNA, 21 nt, Homo sapiens
  source 1..21: mol_type = mRNA
agccttctggaaatatggatg

SEQ ID NO: 28 - RNA, 21 nt, Homo sapiens
  source 1..21: mol_type = mRNA
ttctggaaatatggatgacag

SEQ ID NO: 29 - RNA, 21 nt, Homo sapiens
  source 1..21: mol_type = mRNA
tggaaatatggatgacagtgg

SEQ ID NO: 30 - RNA, 21 nt, Homo sapiens
  source 1..21: mol_type = mRNA
tatggatgacagtggtttttt

SEQ ID NO: 31 - RNA, 21 nt, Homo sapiens
  source 1..21: mol_type = mRNA
tgacagtggttttttctctat

SEQ ID NO: 32 - RNA, 21 nt, Homo sapiens
  source 1..21: mol_type = mRNA
cagtggttttttctctattca

SEQ ID NO: 33 - RNA, 21 nt, Homo sapiens
  source 1..21: mol_type = mRNA
agtggttttttctctattcag

SEQ ID NO: 34 - RNA, 21 nt, Homo sapiens
  source 1..21: mol_type = mRNA
gttttttctctattcaggtta

SEQ ID NO: 35 - RNA, 21 nt, Homo sapiens
  source 1..21: mol_type = mRNA
attcaggttataagcaatgcc

SEQ ID NO: 36 - RNA, 21 nt, Homo sapiens
  source 1..21: mol_type = mRNA
ttcaggttataagcaatgcct

SEQ ID NO: 37 - RNA, 21 nt, Homo sapiens
  source 1..21: mol_type = mRNA
tcaggttataagcaatgcctt

SEQ ID NO: 38 - RNA, 21 nt, Homo sapiens
  source 1..21: mol_type = mRNA
caggttataagcaatgccttg

SEQ ID NO: 39 - RNA, 21 nt, Homo sapiens
  source 1..21: mol_type = mRNA
aggttataagcaatgccttga

SEQ ID NO: 40 - RNA, 21 nt, Homo sapiens
  source 1..21: mol_type = mRNA
ttataagcaatgccttgaaag

SEQ ID NO: 41 - RNA, 21 nt, Homo sapiens
  source 1..21: mol_type = mRNA
tataagcaatgccttgaaagt

SEQ ID NO: 42 - RNA, 21 nt, Homo sapiens
  source 1..21: mol_type = mRNA
tttagaactaatcctgttcaa

SEQ ID NO: 43 - RNA, 21 nt, Homo sapiens
  source 1..21: mol_type = mRNA
ttagaactaatcctgttcaac

SEQ ID NO: 44 - RNA, 21 nt, Homo sapiens
  source 1..21: mol_type = mRNA
tagaactaatcctgttcaaca

SEQ ID NO: 45 - RNA, 21 nt, Homo sapiens
  source 1..21: mol_type = mRNA
actaatcctgttcaacagtcc

SEQ ID NO: 46 - RNA, 21 nt, Homo sapiens
  source 1..21: mol_type = mRNA
ctaatcctgttcaacagtcca

SEQ ID NO: 47 - RNA, 21 nt, Homo sapiens
  source 1..21: mol_type = mRNA
cctgttcaacagtccagagta

SEQ ID NO: 48 - RNA, 21 nt, Homo sapiens
  source 1..21: mol_type = mRNA
ccagagtatcagaggctcagg

SEQ ID NO: 49 - RNA, 21 nt, Homo sapiens
  source 1..21: mol_type = mRNA
tatgcaattataaggaacact

SEQ ID NO: 50 - RNA, 21 nt, Homo sapiens
  source 1..21: mol_type = mRNA
atgcaattataaggaacactg

SEQ ID NO: 51 - RNA, 21 nt, Homo sapiens
  source 1..21: mol_type = mRNA
tgcaattataaggaacactgg

SEQ ID NO: 52 - RNA, 21 nt, Homo sapiens
  source 1..21: mol_type = mRNA
caattataaggaacactggtt

SEQ ID NO: 53 - RNA, 21 nt, Homo sapiens
  source 1..21: mol_type = mRNA
aattataaggaacactggttt

SEQ ID NO: 54 - RNA, 21 nt, Homo sapiens
  source 1..21: mol_type = mRNA
ttataaggaacactggtttac

SEQ ID NO: 55 - RNA, 21 nt, Homo sapiens
  source 1..21: mol_type = mRNA
tataaggaacactggtttaca

SEQ ID NO: 56 - RNA, 21 nt, Homo sapiens
  source 1..21: mol_type = mRNA
ataaggaacactggtttacag

SEQ ID NO: 57 - RNA, 21 nt, Homo sapiens
  source 1..21: mol_type = mRNA
taaggaacactggtttacagt

SEQ ID NO: 58 - RNA, 21 nt, Homo sapiens
  source 1..21: mol_type = mRNA
aggaacactggtttacagtta

SEQ ID NO: 59 - RNA, 21 nt, Homo sapiens
  source 1..21: mol_type = mRNA
ggaacactggtttacagttag

SEQ ID NO: 60 - RNA, 21 nt, Homo sapiens
  source 1..21: mol_type = mRNA
gaacactggtttacagttaga

SEQ ID NO: 61 - RNA, 21 nt, Homo sapiens
  source 1..21: mol_type = mRNA
acactggtttacagttagaaa

SEQ ID NO: 62 - RNA, 21 nt, Homo sapiens
  source 1..21: mol_type = mRNA
agaaaattaggaaaacagtgg

SEQ ID NO: 63 - RNA, 21 nt, Homo sapiens
  source 1..21: mol_type = mRNA
gaaaattaggaaaacagtggt

SEQ ID NO: 64 - RNA, 21 nt, Homo sapiens
  source 1..21: mol_type = mRNA
aaaattaggaaaacagtggtt

SEQ ID NO: 65 - RNA, 21 nt, Homo sapiens
  source 1..21: mol_type = mRNA
aggaaaacagtggtttaactt

SEQ ID NO: 66 - RNA, 21 nt, Homo sapiens
  source 1..21: mol_type = mRNA
ggaaaacagtggtttaacttg

SEQ ID NO: 67 - RNA, 21 nt, Homo sapiens
  source 1..21: mol_type = mRNA
gaaaacagtggtttaacttga

SEQ ID NO: 68 - RNA, 21 nt, Homo sapiens
  source 1..21: mol_type = mRNA
aaacagtggtttaacttgaat

SEQ ID NO: 69 - RNA, 21 nt, Homo sapiens
  source 1..21: mol_type = mRNA
aacagtggtttaacttgaatt

SEQ ID NO: 70 - RNA, 21 nt, Homo sapiens
  source 1..21: mol_type = mRNA
tctctcttgacgggtccagaa

SEQ ID NO: 71 - RNA, 21 nt, Homo sapiens
  source 1..21: mol_type = mRNA
atatcagatacatatcttgca

SEQ ID NO: 72 - RNA, 21 nt, Homo sapiens
  source 1..21: mol_type = mRNA
atcagatacatatcttgcact

SEQ ID NO: 73 - RNA, 21 nt, Homo sapiens
  source 1..21: mol_type = mRNA
acttttcttggctcaattaca

SEQ ID NO: 74 - RNA, 21 nt, Homo sapiens
  source 1..21: mol_type = mRNA
tcttggctcaattacaacagg

SEQ ID NO: 75 - RNA, 21 nt, Homo sapiens
  source 1..21: mol_type = mRNA
cttggctcaattacaacagga

SEQ ID NO: 76 - RNA, 21 nt, Homo sapiens
  source 1..21: mol_type = mRNA
ttggctcaattacaacaggaa

SEQ ID NO: 77 - RNA, 21 nt, Homo sapiens
  source 1..21: mol_type = mRNA
tggctcaattacaacaggaag

SEQ ID NO: 78 - RNA, 21 nt, Homo sapiens
  source 1..21: mol_type = mRNA
ggctcaattacaacaggaagg

SEQ ID NO: 79 - RNA, 21 nt, Homo sapiens
  source 1..21: mol_type = mRNA
caattacaacaggaaggttat

SEQ ID NO: 80 - RNA, 21 nt, Homo sapiens
  source 1..21: mol_type = mRNA
aattacaacaggaaggttatt

SEQ ID NO: 81 - RNA, 21 nt, Homo sapiens
  source 1..21: mol_type = mRNA
caacaggaaggttattctata

SEQ ID NO: 82 - RNA, 21 nt, Homo sapiens
  source 1..21: mol_type = mRNA
acaggaaggttattctatatt

SEQ ID NO: 83 - RNA, 21 nt, Homo sapiens
  source 1..21: mol_type = mRNA
caggaaggttattctatattt

SEQ ID NO: 84 - RNA, 21 nt, Homo sapiens
  source 1..21: mol_type = mRNA
tctgccagattgcgaagctga

SEQ ID NO: 85 - RNA, 21 nt, Homo sapiens
  source 1..21: mol_type = mRNA
atgcatcgaccaaaacttatt

SEQ ID NO: 86 - RNA, 21 nt, Homo sapiens
  source 1..21: mol_type = mRNA
gcatcgaccaaaacttattgg

SEQ ID NO: 87 - RNA, 21 nt, Homo sapiens
  source 1..21: mol_type = mRNA
tcgaccaaaacttattggaga

SEQ ID NO: 88 - RNA, 21 nt, Homo sapiens
  source 1..21: mol_type = mRNA
ccaaaacttattggagaagaa

SEQ ID NO: 89 - RNA, 21 nt, Homo sapiens
  source 1..21: mol_type = mRNA
acttattggagaagaattagc

SEQ ID NO: 90 - RNA, 21 nt, Homo sapiens
  source 1..21: mol_type = mRNA
tattggagaagaattagcaca

SEQ ID NO: 91 - RNA, 21 nt, Homo sapiens
  source 1..21: mol_type = mRNA
aaagagcaaagagtccataaa

SEQ ID NO: 92 - RNA, 21 nt, Homo sapiens
  source 1..21: mol_type = mRNA
agagtccataaaacagacctg

SEQ ID NO: 93 - RNA, 21 nt, Homo sapiens
  source 1..21: mol_type = mRNA
tccataaaacagacctggaac

SEQ ID NO: 94 - RNA, 21 nt, Homo sapiens
  source 1..21: mol_type = mRNA
tagaagcaaatgatggctcag

SEQ ID NO: 95 - RNA, 21 nt, Homo sapiens
  source 1..21: mol_type = mRNA
aatgatggctcaggaatgtta

SEQ ID NO: 96 - RNA, 21 nt, Homo sapiens
  source 1..21: mol_type = mRNA
gcagagggctctggcactaag

SEQ ID NO: 97 - RNA, 21 nt, Homo sapiens
  source 1..21: mol_type = mRNA
ctaagtcgccaagaaattgac

SEQ ID NO: 98 - RNA, 21 nt, Homo sapiens
  source 1..21: mol_type = mRNA
ttcagctaagtatgcaaggta

SEQ ID NO: 99 - RNA, 21 nt, Homo sapiens
  source 1..21: mol_type = mRNA
tcagctaagtatgcaaggtag

SEQ ID NO: 100 - RNA, 21 nt, Homo sapiens
  source 1..21: mol_type = mRNA
gctaagtatgcaaggtagttc

SEQ ID NO: 101 - RNA, 21 nt, Homo sapiens
  source 1..21: mol_type = mRNA
ctaagtatgcaaggtagttcc

SEQ ID NO: 102 - RNA, 21 nt, Homo sapiens
  source 1..21: mol_type = mRNA
taagtatgcaaggtagttcca

SEQ ID NO: 103 - RNA, 21 nt, Homo sapiens
  source 1..21: mol_type = mRNA
aagtatgcaaggtagttccag

SEQ ID NO: 104 - RNA, 21 nt, Homo sapiens
  source 1..21: mol_type = mRNA
agtatgcaaggtagttccaga

SEQ ID NO: 105 - RNA, 21 nt, Homo sapiens
  source 1..21: mol_type = mRNA
gtatgcaaggtagttccagaa

SEQ ID NO: 106 - RNA, 21 nt, Homo sapiens
  source 1..21: mol_type = mRNA
atgcaaggtagttccagaaac

SEQ ID NO: 107 - RNA, 21 nt, Homo sapiens
  source 1..21: mol_type = mRNA
atgacacagacatcaggtaca

SEQ ID NO: 108 - RNA, 21 nt, Homo sapiens
  source 1..21: mol_type = mRNA
gacacagacatcaggtacaaa

SEQ ID NO: 109 - RNA, 21 nt, Homo sapiens
  source 1..21: mol_type = mRNA
cacagacatcaggtacaaatc

SEQ ID NO: 110 - RNA, 21 nt, Homo sapiens
  source 1..21: mol_type = mRNA
tcaggtacaaatcttacttca

SEQ ID NO: 111 - RNA, 21 nt, Homo sapiens
  source 1..21: mol_type = mRNA
tcttacttcagaagagcttcg

SEQ ID NO: 112 - RNA, 21 nt, Homo sapiens
  source 1..21: mol_type = mRNA
ttacttcagaagagcttcgga

SEQ ID NO: 113 - RNA, 21 nt, Homo sapiens
  source 1..21: mol_type = mRNA
tacttcagaagagcttcggaa

SEQ ID NO: 114 - RNA, 21 nt, Homo sapiens
  source 1..21: mol_type = mRNA
tcagaagagcttcggaagaga

SEQ ID NO: 115 - RNA, 21 nt, Homo sapiens
  source 1..21: mol_type = mRNA
gcttcggaagagacgagaagc

SEQ ID NO: 116 - RNA, 21 nt, Homo sapiens
  source 1..21: mol_type = mRNA
cttcggaagagacgagaagcc

SEQ ID NO: 117 - RNA, 21 nt, Homo sapiens
  source 1..21: mol_type = mRNA
ggaagagacgagaagcctact

SEQ ID NO: 118 - RNA, 21 nt, Homo sapiens
  source 1..21: mol_type = mRNA
gaagagacgagaagcctactt

SEQ ID NO: 119 - RNA, 21 nt, Homo sapiens
  source 1..21: mol_type = mRNA
aagagacgagaagcctacttt

SEQ ID NO: 120 - RNA, 21 nt, Homo sapiens
  source 1..21: mol_type = mRNA
agagacgagaagcctactttg

SEQ ID NO: 121 - RNA, 21 nt, Homo sapiens
  source 1..21: mol_type = mRNA
gagacgagaagcctactttga

SEQ ID NO: 122 - RNA, 21 nt, Homo sapiens
  source 1..21: mol_type = mRNA
agacgagaagcctactttgaa

SEQ ID NO: 123 - RNA, 21 nt, Homo sapiens
  source 1..21: mol_type = mRNA
agaagcctactttgaaaaaca

SEQ ID NO: 124 - RNA, 21 nt, Homo sapiens
  source 1..21: mol_type = mRNA
acttgggagtgatctaggtga

SEQ ID NO: 125 - RNA, 21 nt, Homo sapiens
  source 1..21: mol_type = mRNA
cttgggagtgatctaggtgat

SEQ ID NO: 126 - RNA, 21 nt, Homo sapiens
  source 1..21: mol_type = mRNA
ttgggagtgatctaggtgatg

SEQ ID NO: 127 - RNA, 21 nt, Homo sapiens
  source 1..21: mol_type = mRNA
gtgatctaggtgatgctatga

SEQ ID NO: 128 - RNA, 21 nt, Homo sapiens
  source 1..21: mol_type = mRNA
caggcagctgtgaccatgtct

SEQ ID NO: 129 - RNA, 21 nt, Homo sapiens
  source 1..21: mol_type = mRNA
gcagctgtgaccatgtcttta

SEQ ID NO: 130 - RNA, 21 nt, Homo sapiens
  source 1..21: mol_type = mRNA
cagctgtgaccatgtctttag

SEQ ID NO: 131 - RNA, 21 nt, Homo sapiens
  source 1..21: mol_type = mRNA
agctgtgaccatgtctttaga

SEQ ID NO: 132 - RNA, 21 nt, Homo sapiens
  source 1..21: mol_type = mRNA
gctgtgaccatgtctttagaa

SEQ ID NO: 133 - RNA, 21 nt, Homo sapiens
  source 1..21: mol_type = mRNA
ctgtgaccatgtctttagaaa

SEQ ID NO: 134 - RNA, 21 nt, Homo sapiens
  source 1..21: mol_type = mRNA
gaccatgtctttagaaactgt

SEQ ID NO: 135 - RNA, 21 nt, Homo sapiens
  source 1..21: mol_type = mRNA
ccatgtctttagaaactgtca

SEQ ID NO: 136 - RNA, 21 nt, Homo sapiens
  source 1..21: mol_type = mRNA
tgtctttagaaactgtcagaa

SEQ ID NO: 137 - RNA, 21 nt, Homo sapiens
  source 1..21: mol_type = mRNA
aactgtcagaaatgatttgaa

SEQ ID NO: 138 - RNA, 21 nt, Homo sapiens
  source 1..21: mol_type = mRNA
atcctgtgtgattacagcata

SEQ ID NO: 139 - RNA, 21 nt, Homo sapiens
  source 1..21: mol_type = mRNA
tcctgtgtgattacagcatag

SEQ ID NO: 140 - RNA, 21 nt, Homo sapiens
  source 1..21: mol_type = mRNA
cctgtgtgattacagcatagg

SEQ ID NO: 141 - RNA, 21 nt, Homo sapiens
  source 1..21: mol_type = mRNA
tacagcatagggtccactttg

SEQ ID NO: 142 - RNA, 21 nt, Homo sapiens
  source 1..21: mol_type = mRNA
agggtccactttggtaatgtg

SEQ ID NO: 143 - RNA, 21 nt, Homo sapiens
  source 1..21: mol_type = mRNA
gtccactttggtaatgtgtca

SEQ ID NO: 144 - RNA, 21 nt, Homo sapiens
  source 1..21: mol_type = mRNA
tccactttggtaatgtgtcaa

SEQ ID NO: 145 - RNA, 21 nt, Homo sapiens
  source 1..21: mol_type = mRNA
ccactttggtaatgtgtcaaa

SEQ ID NO: 146 - RNA, 21 nt, Homo sapiens
  source 1..21: mol_type = mRNA
tttggtaatgtgtcaaagaga

SEQ ID NO: 147 - RNA, 21 nt, Homo sapiens
  source 1..21: mol_type = mRNA
gtaatgtgtcaaagagatgag

SEQ ID NO: 148 - RNA, 21 nt, Homo sapiens
  source 1..21: mol_type = mRNA
taatgtgtcaaagagatgagg

SEQ ID NO: 149 - RNA, 21 nt, Homo sapiens
  source 1..21: mol_type = mRNA
tagcggtttgcaaacaaaatg

SEQ ID NO: 150 - RNA, 21 nt, Homo sapiens
  source 1..21: mol_type = mRNA
gtttgcaaacaaaatgatggg

SEQ ID NO: 151 - RNA, 21 nt, Homo sapiens
  source 1..21: mol_type = mRNA
tttgcaaacaaaatgatggga

SEQ ID NO: 152 - RNA, 21 nt, Homo sapiens
  source 1..21: mol_type = mRNA
gtcggttgtaggactaaataa

SEQ ID NO: 153 - RNA, 21 nt, Homo sapiens
  source 1..21: mol_type = mRNA
gttgtaggactaaataatgat

SEQ ID NO: 154 - RNA, 21 nt, Homo sapiens
  source 1..21: mol_type = mRNA
gtaggactaaataatgatctt

SEQ ID NO: 155 - RNA, 21 nt, Homo sapiens
  source 1..21: mol_type = mRNA
aggactaaataatgatcttcc

SEQ ID NO: 156 - RNA, 21 nt, Homo sapiens
  source 1..21: mol_type = mRNA
tccaaatattagccaaagagg

SEQ ID NO: 157 - RNA, 21 nt, Homo sapiens
  source 1..21: mol_type = mRNA
aaatattagccaaagaggcat

SEQ ID NO: 158 - RNA, 21 nt, Homo sapiens
  source 1..21: mol_type = mRNA
attagccaaagaggcattcag

SEQ ID NO: 159 - RNA, 21 nt, Homo sapiens
  source 1..21: mol_type = mRNA
ttagccaaagaggcattcagc

SEQ ID NO: 160 - RNA, 21 nt, Homo sapiens
  source 1..21: mol_type = mRNA
gccaaagaggcattcagcaat

SEQ ID NO: 161 - RNA, 21 nt, Homo sapiens
  source 1..21: mol_type = mRNA
aagaggcattcagcaattaaa

SEQ ID NO: 162 - RNA, 21 nt, Homo sapiens
  source 1..21: mol_type = mRNA
agaggcattcagcaattaaag

SEQ ID NO: 163 - RNA, 21 nt, Homo sapiens
  source 1..21: mol_type = mRNA
ggcattcagcaattaaagaca

SEQ ID NO: 164 - RNA, 21 nt, Homo sapiens
  source 1..21: mol_type = mRNA
ctttttcttttttgagtgtgc

SEQ ID NO: 165 - RNA, 21 nt, Homo sapiens
  source 1..21: mol_type = mRNA
tcttttttgagtgtgcaatat

SEQ ID NO: 166 - RNA, 21 nt, Homo sapiens
  source 1..21: mol_type = mRNA
cttttttgagtgtgcaatatg

SEQ ID NO: 167 - RNA, 21 nt, Homo sapiens
  source 1..21: mol_type = mRNA
ttagggcatttttcttggatc

SEQ ID NO: 168 - RNA, 21 nt, Homo sapiens
  source 1..21: mol_type = mRNA
ccaaattaacctttcaggaaa

SEQ ID NO: 169 - RNA, 21 nt, Homo sapiens
  source 1..21: mol_type = mRNA
ttaacctttcaggaaagtatc

SEQ ID NO: 170 - RNA, 21 nt, Homo sapiens
  source 1..21: mol_type = mRNA
taacctttcaggaaagtatct

SEQ ID NO: 171 - RNA, 21 nt, Homo sapiens
  source 1..21: mol_type = mRNA
gatgttgataatagtaatggt

SEQ ID NO: 172 - RNA, 21 nt, Homo sapiens
  source 1..21: mol_type = mRNA
gataatagtaatggttctaga

SEQ ID NO: 173 - RNA, 21 nt, Homo sapiens
  source 1..21: mol_type = mRNA
ttctcccttcaactgtatacc

SEQ ID NO: 174 - RNA, 21 nt, Homo sapiens
  source 1..21: mol_type = mRNA
ccgtgtgctccagtgttttct

SEQ ID NO: 175 - RNA, 21 nt, Homo sapiens
  source 1..21: mol_type = mRNA
tcattattttcccacaattct

SEQ ID NO: 176 - RNA, 21 nt, Homo sapiens
  source 1..21: mol_type = mRNA
tttcccacaattcttttgaaa

SEQ ID NO: 177 - RNA, 21 nt, Homo sapiens
  source 1..21: mol_type = mRNA
tcccacaattcttttgaaaga

SEQ ID NO: 178 - RNA, 21 nt, Homo sapiens
  source 1..21: mol_type = mRNA
tgaaagatggtaatcttttct

SEQ ID NO: 179 - RNA, 21 nt, Homo sapiens
  source 1..21: mol_type = mRNA
ccctacgatgggatcattatt

SEQ ID NO: 180 - RNA, 21 nt, Homo sapiens
  source 1..21: mol_type = mRNA
cgatgggatcattatttcatg

SEQ ID NO: 181 - RNA, 21 nt, Homo sapiens
  source 1..21: mol_type = mRNA
tgggatcattatttcatgact

SEQ ID NO: 182 - RNA, 21 nt, Homo sapiens
  source 1..21: mol_type = mRNA
agtacttgagaataaatgagc

SEQ ID NO: 183 - RNA, 21 nt, Homo sapiens
  source 1..21: mol_type = mRNA
tctaggtaaggcctgctcacc

SEQ ID NO: 184 - RNA, 21 nt, Homo sapiens
  source 1..21: mol_type = mRNA
ttcatcatgttcgctaccttc

SEQ ID NO: 185 - RNA, 21 nt, Homo sapiens
  source 1..21: mol_type = mRNA
atcatgttcgctaccttcaca

SEQ ID NO: 186 - RNA, 21 nt, Homo sapiens
  source 1..21: mol_type = mRNA
tcatgttcgctaccttcacac

SEQ ID NO: 187 - RNA, 21 nt, Homo sapiens
  source 1..21: mol_type = mRNA
tgttcgctaccttcacacttt

SEQ ID NO: 188 - RNA, 21 nt, Homo sapiens
  source 1..21: mol_type = mRNA
ttcgctaccttcacactttat

SEQ ID NO: 189 - RNA, 21 nt, Homo sapiens
  source 1..21: mol_type = mRNA
tttatctgacatacgagctcc

SEQ ID NO: 190 - RNA, 21 nt, Homo sapiens
  source 1..21: mol_type = mRNA
agctccatgtgatttttgctt

SEQ ID NO: 191 - RNA, 21 nt, Homo sapiens
  source 1..21: mol_type = mRNA
gctccatgtgatttttgcttt

SEQ ID NO: 192 - RNA, 21 nt, Homo sapiens
  source 1..21: mol_type = mRNA
taaatttccctggattaagga

SEQ ID NO: 193 - RNA, 16 nt, Synthetic construct
  source 1..16: mol_type = other RNA
agaaggctcactttga

SEQ ID NO: 194 - RNA, 16 nt, Synthetic construct
  source 1..16: mol_type = other RNA
gaaggctcactttgta

SEQ ID NO: 195 - RNA, 16 nt, Synthetic construct
  source 1..16: mol_type = other RNA
aaggctcactttgtga

SEQ ID NO: 196 - RNA, 16 nt, Synthetic construct
  source 1..16: mol_type = other RNA
aggctcactttgtgca

SEQ ID NO: 197 - RNA, 16 nt, Synthetic construct
  source 1..16: mol_type = other RNA
gctcactttgtgctca

SEQ ID NO: 198 - RNA, 16 nt, Synthetic construct
  source 1..16: mol_type = other RNA
tgtgctcaacattgca

SEQ ID NO: 199 - RNA, 16 nt, Synthetic construct
  source 1..16: mol_type = other RNA
acattgcctgaataaa

SEQ ID NO: 200 - RNA, 16 nt, Synthetic construct
  source 1..16: mol_type = other RNA
tattgcaaggagaata

SEQ ID NO: 201 - RNA, 16 nt, Synthetic construct
  source 1..16: mol_type = other RNA
gccctgtggaattata

SEQ ID NO: 202 - RNA, 16 nt, Synthetic construct
  source 1..16: mol_type = other RNA
ccctgtggaattatca

SEQ ID NO: 203 - RNA, 16 nt, Synthetic construct
  source 1..16: mol_type = other RNA
ctgtggaattatccta

SEQ ID NO: 204 - RNA, 16 nt, Synthetic construct
  source 1..16: mol_type = other RNA
attatcctcaattgca

SEQ ID NO: 205 - RNA, 16 nt, Synthetic construct
  source 1..16: mol_type = other RNA
tatcctcaattgcaca

SEQ ID NO: 206 - RNA, 16 nt, Synthetic construct
  source 1..16: mol_type = other RNA
ggaggagaggatgaga

SEQ ID NO: 207 - RNA, 16 nt, Synthetic construct
  source 1..16: mol_type = other RNA
agaaggaggagttaca

SEQ ID NO: 208 - RNA, 16 nt, Synthetic construct
  source 1..16: mol_type = other RNA
aggaggagttactaga

SEQ ID NO: 209 - RNA, 16 nt, Synthetic construct
  source 1..16: mol_type = other RNA
aggagttactagtgaa

SEQ ID NO: 210 - RNA, 16 nt, Synthetic construct
  source 1..16: mol_type = other RNA
actagtgaagattata

SEQ ID NO: 211 - RNA, 16 nt, Synthetic construct
  source 1..16: mol_type = other RNA
ctagtgaagattatca

SEQ ID NO: 212 - RNA, 16 nt, Synthetic construct
  source 1..16: mol_type = other RNA
agtgaagattatcgca

SEQ ID NO: 213 - RNA, 16 nt, Synthetic construct
  source 1..16: mol_type = other RNA
gcacgtttttacagca

SEQ ID NO: 214 - RNA, 16 nt, Synthetic construct
  source 1..16: mol_type = other RNA
ttacagcagccttcta

SEQ ID NO: 215 - RNA, 16 nt, Synthetic construct
  source 1..16: mol_type = other RNA
tacagcagccttctga

SEQ ID NO: 216 - RNA, 16 nt, Synthetic construct
  source 1..16: mol_type = other RNA
acagcagccttctgga

SEQ ID NO: 217 - RNA, 16 nt, Synthetic construct
  source 1..16: mol_type = other RNA
agccttctggaaataa

SEQ ID NO: 218 - RNA, 16 nt, Synthetic construct
  source 1..16: mol_type = other RNA
cttctggaaatatgga

SEQ ID NO: 219 - RNA, 16 nt, Synthetic construct
  source 1..16: mol_type = other RNA
tctggaaatatggata

SEQ ID NO: 220 - RNA, 16 nt, Synthetic construct
  source 1..16: mol_type = other RNA
gaaatatggatgacaa

SEQ ID NO: 221 - RNA, 16 nt, Synthetic construct
  source 1..16: mol_type = other RNA
atatggatgacagtga

SEQ ID NO: 222 - RNA, 16 nt, Synthetic construct
  source 1..16: mol_type = other RNA
atgacagtggttttta

SEQ ID NO: 223 - RNA, 16 nt, Synthetic construct
  source 1..16: mol_type = other RNA
gtggttttttctctaa

SEQ ID NO: 224 - RNA, 16 nt, Synthetic construct
  source 1..16: mol_type = other RNA
gttttttctctattca

SEQ ID NO: 225 - RNA, 16 nt, Synthetic construct
  source 1..16: mol_type = other RNA
ttttttctctattcaa

SEQ ID NO: 226 - RNA, 16 nt, Synthetic construct
  source 1..16: mol_type = other RNA
ttctctattcaggtta

SEQ ID NO: 227 - RNA, 16 nt, Synthetic construct
  source 1..16: mol_type = other RNA
ggttataagcaatgca

SEQ ID NO: 228 - RNA, 16 nt, Synthetic construct
  source 1..16: mol_type = other RNA
gttataagcaatgcca

SEQ ID NO: 229 - RNA, 16 nt, Synthetic construct
  source 1..16: mol_type = other RNA
ttataagcaatgccta

SEQ ID NO: 230 - RNA, 16 nt, Synthetic construct
  source 1..16: mol_type = other RNA
tataagcaatgcctta

SEQ ID NO: 231 - RNA, 16 nt, Synthetic construct
  source 1..16: mol_type = other RNA
ataagcaatgccttga

SEQ ID NO: 232 - RNA, 16 nt, Synthetic construct
  source 1..16: mol_type = other RNA
agcaatgccttgaaaa

SEQ ID NO: 233 - RNA, 16 nt, Synthetic construct
  source 1..16: mol_type = other RNA
gcaatgccttgaaaga

SEQ ID NO: 234 - RNA, 16 nt, Synthetic construct
  source 1..16: mol_type = other RNA
aactaatcctgttcaa

SEQ ID NO: 235 - RNA, 16 nt, Synthetic construct
  source 1..16: mol_type = other RNA
actaatcctgttcaaa

SEQ ID NO: 236 - RNA, 16 nt, Synthetic construct
  source 1..16: mol_type = other RNA
ctaatcctgttcaaca

SEQ ID NO: 237 - RNA, 16 nt, Synthetic construct
  source 1..16: mol_type = other RNA
tcctgttcaacagtca

SEQ ID NO: 238 - RNA, 16 nt, Synthetic construct
  source 1..16: mol_type = other RNA
cctgttcaacagtcca

SEQ ID NO: 239 - RNA, 16 nt, Synthetic construct
  source 1..16: mol_type = other RNA
tcaacagtccagagta

SEQ ID NO: 240 - RNA, 16 nt, Synthetic construct
  source 1..16: mol_type = other RNA
gtatcagaggctcaga

SEQ ID NO: 241 - RNA, 16 nt, Synthetic construct
  source 1..16: mol_type = other RNA
aattataaggaacaca

SEQ ID NO: 242 - RNA, 16 nt, Synthetic construct
  source 1..16: mol_type = other RNA
attataaggaacacta

SEQ ID NO: 243 - RNA, 16 nt, Synthetic construct
  source 1..16: mol_type = other RNA
ttataaggaacactga

SEQ ID NO: 244 - RNA, 16 nt, Synthetic construct
  source 1..16: mol_type = other RNA
ataaggaacactggta

SEQ ID NO: 245 - RNA, 16 nt, Synthetic construct
  source 1..16: mol_type = other RNA
taaggaacactggtta

SEQ ID NO: 246 - RNA, 16 nt, Synthetic construct
  source 1..16: mol_type = other RNA
aggaacactggtttaa

SEQ ID NO: 247 - RNA, 16 nt, Synthetic construct
  source 1..16: mol_type = other RNA
ggaacactggtttaca

SEQ ID NO: 248 - RNA, 16 nt, Synthetic construct
  source 1..16: mol_type = other RNA
gaacactggtttacaa

SEQ ID NO: 249 - RNA, 16 nt, Synthetic construct
  source 1..16: mol_type = other RNA
aacactggtttacaga

SEQ ID NO: 250 - RNA, 16 nt, Synthetic construct
  source 1..16: mol_type = other RNA
cactggtttacagtta

SEQ ID NO: 251 - RNA, 16 nt, Synthetic construct
  source 1..16: mol_type = other RNA
actggtttacagttaa

SEQ ID NO: 252 - RNA, 16 nt, Synthetic construct
  source 1..16: mol_type = other RNA
ctggtttacagttaga

SEQ ID NO: 253 - RNA, 16 nt, Synthetic construct
  source 1..16: mol_type = other RNA
ggtttacagttagaaa

SEQ ID NO: 254 - RNA, 16 nt, Synthetic construct
  source 1..16: mol_type = other RNA
attaggaaaacagtga

SEQ ID NO: 255 - RNA, 16 nt, Synthetic construct
  source 1..16: mol_type = other RNA
ttaggaaaacagtgga

SEQ ID NO: 256 - RNA, 16 nt, Synthetic construct
  source 1..16: mol_type = other RNA
taggaaaacagtggta

SEQ ID NO: 257 - RNA, 16 nt, Synthetic construct
  source 1..16: mol_type = other RNA
aacagtggtttaacta

SEQ ID NO: 258 - RNA, 16 nt, Synthetic construct
  source 1..16: mol_type = other RNA
acagtggtttaactta

SEQ ID NO: 259 - RNA, 16 nt, Synthetic construct
  source 1..16: mol_type = other RNA
cagtggtttaacttga

SEQ ID NO: 260 - RNA, 16 nt, Synthetic construct
  source 1..16: mol_type = other RNA
gtggtttaacttgaaa

SEQ ID NO: 261 - RNA, 16 nt, Synthetic construct
  source 1..16: mol_type = other RNA
tggtttaacttgaata

SEQ ID NO: 262 - RNA, 16 nt, Synthetic construct
  source 1..16: mol_type = other RNA
cttgacgggtccagaa

SEQ ID NO: 263 - RNA, 16 nt, Synthetic construct
  source 1..16: mol_type = other RNA
agatacatatcttgca

SEQ ID NO: 264 - RNA, 16 nt, Synthetic construct
  source 1..16: mol_type = other RNA
atacatatcttgcaca

SEQ ID NO: 265 - RNA, 16 nt, Synthetic construct
  source 1..16: mol_type = other RNA
tcttggctcaattaca

SEQ ID NO: 266 - RNA, 16 nt, Synthetic construct
  source 1..16: mol_type = other RNA
gctcaattacaacaga

SEQ ID NO: 267 - RNA, 16 nt, Synthetic construct
  source 1..16: mol_type = other RNA
ctcaattacaacagga

SEQ ID NO: 268 - RNA, 16 nt, Synthetic construct
  source 1..16: mol_type = other RNA
tcaattacaacaggaa

SEQ ID NO: 269 - RNA, 16 nt, Synthetic construct
  source 1..16: mol_type = other RNA
caattacaacaggaaa

SEQ ID NO: 270 - RNA, 16 nt, Synthetic construct
  source 1..16: mol_type = other RNA
aattacaacaggaaga

SEQ ID NO: 271 - RNA, 16 nt, Synthetic construct
  source 1..16: mol_type = other RNA
acaacaggaaggttaa

SEQ ID NO: 272 - RNA, 16 nt, Synthetic construct
  source 1..16: mol_type = other RNA
caacaggaaggttata

SEQ ID NO: 273 - RNA, 16 nt, Synthetic construct
  source 1..16: mol_type = other RNA
ggaaggttattctata

SEQ ID NO: 274 - RNA, 16 nt, Synthetic construct
  source 1..16: mol_type = other RNA
aaggttattctatata

SEQ ID NO: 275 - RNA, 16 nt, Synthetic construct
  source 1..16: mol_type = other RNA
aggttattctatatta

SEQ ID NO: 276 - RNA, 16 nt, Synthetic construct
  source 1..16: mol_type = other RNA
cagattgcgaagctga

SEQ ID NO: 277 - RNA, 16 nt, Synthetic construct
  source 1..16: mol_type = other RNA
tcgaccaaaacttata

SEQ ID NO: 278 - RNA, 16 nt, Synthetic construct
  source 1..16: mol_type = other RNA
gaccaaaacttattga

SEQ ID NO: 279 - RNA, 16 nt, Synthetic construct
  source 1..16: mol_type = other RNA
caaaacttattggaga

SEQ ID NO: 280 - RNA, 16 nt, Synthetic construct
  source 1..16: mol_type = other RNA
acttattggagaagaa

SEQ ID NO: 281 - RNA, 16 nt, Synthetic construct
  source 1..16: mol_type = other RNA
ttggagaagaattaga

SEQ ID NO: 282 - RNA, 16 nt, Synthetic construct
  source 1..16: mol_type = other RNA
gagaagaattagcaca

SEQ ID NO: 283 - RNA, 16 nt, Synthetic construct
  source 1..16: mol_type = other RNA
gcaaagagtccataaa

SEQ ID NO: 284 - RNA, 16 nt, Synthetic construct
  source 1..16: mol_type = other RNA
ccataaaacagaccta

SEQ ID NO: 285 - RNA, 16 nt, Synthetic construct
  source 1..16: mol_type = other RNA
aaaacagacctggaaa

SEQ ID NO: 286 - RNA, 16 nt, Synthetic construct
  source 1..16: mol_type = other RNA
gcaaatgatggctcaa

SEQ ID NO: 287 - RNA, 16 nt, Synthetic construct
  source 1..16: mol_type = other RNA
tggctcaggaatgtta

SEQ ID NO: 288 - RNA, 16 nt, Synthetic construct
  source 1..16: mol_type = other RNA
gggctctggcactaaa

SEQ ID NO: 289 - RNA, 16 nt, Synthetic construct
  source 1..16: mol_type = other RNA
tcgccaagaaattgaa

SEQ ID NO: 290 - RNA, 16 nt, Synthetic construct
  source 1..16: mol_type = other RNA
ctaagtatgcaaggta

SEQ ID NO: 291 - RNA, 16 nt, Synthetic construct
  source 1..16: mol_type = other RNA
taagtatgcaaggtaa

SEQ ID NO: 292 - RNA, 16 nt, Synthetic construct
  source 1..16: mol_type = other RNA
gtatgcaaggtagtta

SEQ ID NO: 293 - RNA, 16 nt, Synthetic construct
  source 1..16: mol_type = other RNA
tatgcaaggtagttca

SEQ ID NO: 294 - RNA, 16 nt, Synthetic construct
  source 1..16: mol_type = other RNA
atgcaaggtagttcca

SEQ ID NO: 295 - RNA, 16 nt, Synthetic construct
  source 1..16: mol_type = other RNA
tgcaaggtagttccaa

SEQ ID NO: 296 - RNA, 16 nt, Synthetic construct
  source 1..16: mol_type = other RNA
gcaaggtagttccaga

SEQ ID NO: 297 - RNA, 16 nt, Synthetic construct
  source 1..16: mol_type = other RNA
caaggtagttccagaa

SEQ ID NO: 298 - RNA, 16 nt, Synthetic construct
  source 1..16: mol_type = other RNA
aggtagttccagaaaa

SEQ ID NO: 299 - RNA, 16 nt, Synthetic construct
  source 1..16: mol_type = other RNA
acagacatcaggtaca

SEQ ID NO: 300 - RNA, 16 nt, Synthetic construct
  source 1..16: mol_type = other RNA
agacatcaggtacaaa

SEQ ID NO: 301 - RNA, 16 nt, Synthetic construct
  source 1..16: mol_type = other RNA
acatcaggtacaaata

SEQ ID NO: 302 - RNA, 16 nt, Synthetic construct
  source 1..16: mol_type = other RNA
tacaaatcttacttca

SEQ ID NO: 303 - RNA, 16 nt, Synthetic construct
  source 1..16: mol_type = other RNA
cttcagaagagcttca

SEQ ID NO: 304 - RNA, 16 nt, Synthetic construct
  source 1..16: mol_type = other RNA
tcagaagagcttcgga

SEQ ID NO: 305 - RNA, 16 nt, Synthetic construct
  source 1..16: mol_type = other RNA
cagaagagcttcggaa

SEQ ID NO: 306 - RNA, 16 nt, Synthetic construct
  source 1..16: mol_type = other RNA
agagcttcggaagaga

SEQ ID NO: 307 - RNA, 16 nt, Synthetic construct
  source 1..16: mol_type = other RNA
ggaagagacgagaaga

SEQ ID NO: 308 - RNA, 16 nt, Synthetic construct
  source 1..16: mol_type = other RNA
gaagagacgagaagca

SEQ ID NO: 309 - RNA, 16 nt, Synthetic construct
  source 1..16: mol_type = other RNA
agacgagaagcctaca

SEQ ID NO: 310 - RNA, 16 nt, Synthetic construct
  source 1..16: mol_type = other RNA
gacgagaagcctacta

SEQ ID NO: 311 - RNA, 16 nt, Synthetic construct
  source 1..16: mol_type = other RNA
acgagaagcctactta

SEQ ID NO: 312 - RNA, 16 nt, Synthetic construct
  source 1..16: mol_type = other RNA
cgagaagcctacttta

SEQ ID NO: 313 - RNA, 16 nt, Synthetic construct
  source 1..16: mol_type = other RNA
gagaagcctactttga

SEQ ID NO: 314 - RNA, 16 nt, Synthetic construct
  source 1..16: mol_type = other RNA
agaagcctactttgaa

SEQ ID NO: 315 - RNA, 16 nt, Synthetic construct
  source 1..16: mol_type = other RNA
cctactttgaaaaaca

SEQ ID NO: 316 - RNA, 16 nt, Synthetic construct
  source 1..16: mol_type = other RNA
ggagtgatctaggtga

SEQ ID NO: 317 - RNA, 16 nt, Synthetic construct
  source 1..16: mol_type = other RNA
gagtgatctaggtgaa

SEQ ID NO: 318 - RNA, 16 nt, Synthetic construct
  source 1..16: mol_type = other RNA
agtgatctaggtgata

SEQ ID NO: 319 - RNA, 16 nt, Synthetic construct
  source 1..16: mol_type = other RNA
ctaggtgatgctatga

SEQ ID NO: 320 - RNA, 16 nt, Synthetic construct
  source 1..16: mol_type = other RNA
agctgtgaccatgtca

SEQ ID NO: 321 - RNA, 16 nt, Synthetic construct
  source 1..16: mol_type = other RNA
tgtgaccatgtcttta

SEQ ID NO: 322 - RNA, 16 nt, Synthetic construct
  source 1..16: mol_type = other RNA
gtgaccatgtctttaa

SEQ ID NO: 323 - RNA, 16 nt, Synthetic construct
  source 1..16: mol_type = other RNA
tgaccatgtctttaga

SEQ ID NO: 324 - RNA, 16 nt, Synthetic construct
  source 1..16: mol_type = other RNA
gaccatgtctttagaa

SEQ ID NO: 325 - RNA, 16 nt, Synthetic construct
  source 1..16: mol_type = other RNA
accatgtctttagaaa

SEQ ID NO: 326 - RNA, 16 nt, Synthetic construct
  source 1..16: mol_type = other RNA
tgtctttagaaactga

SEQ ID NO: 327 - RNA, 16 nt, Synthetic construct
  source 1..16: mol_type = other RNA
tctttagaaactgtca

SEQ ID NO: 328 - RNA, 16 nt, Synthetic construct
  source 1..16: mol_type = other RNA
ttagaaactgtcagaa

SEQ ID NO: 329 - RNA, 16 nt, Synthetic construct
  source 1..16: mol_type = other RNA
tcagaaatgatttgaa

SEQ ID NO: 330 - RNA, 16 nt, Synthetic construct
  source 1..16: mol_type = other RNA
gtgtgattacagcata

SEQ ID NO: 331 - RNA, 16 nt, Synthetic construct
  source 1..16: mol_type = other RNA
tgtgattacagcataa

SEQ ID NO: 332 - RNA, 16 nt, Synthetic construct
  source 1..16: mol_type = other RNA
gtgattacagcataga

SEQ ID NO: 333 - RNA, 16 nt, Synthetic construct
  source 1..16: mol_type = other RNA
catagggtccacttta

SEQ ID NO: 334 - RNA, 16 nt, Synthetic construct
  source 1..16: mol_type = other RNA
ccactttggtaatgta

SEQ ID NO: 335 - RNA, 16 nt, Synthetic construct
  source 1..16: mol_type = other RNA
ctttggtaatgtgtca

SEQ ID NO: 336 - RNA, 16 nt, Synthetic construct
  source 1..16: mol_type = other RNA
tttggtaatgtgtcaa

SEQ ID NO: 337 - RNA, 16 nt, Synthetic construct
  source 1..16: mol_type = other RNA
ttggtaatgtgtcaaa

SEQ ID NO: 338 - RNA, 16 nt, Synthetic construct
  source 1..16: mol_type = other RNA
taatgtgtcaaagaga

SEQ ID NO: 339 - RNA, 16 nt, Synthetic construct
  source 1..16: mol_type = other RNA
gtgtcaaagagatgaa

SEQ ID NO: 340 - RNA, 16 nt, Synthetic construct
  source 1..16: mol_type = other RNA
tgtcaaagagatgaga

SEQ ID NO: 341 - RNA, 16 nt, Synthetic construct
  source 1..16: mol_type = other RNA
gtttgcaaacaaaata

SEQ ID NO: 342 - RNA, 16 nt, Synthetic construct
  source 1..16: mol_type = other RNA
caaacaaaatgatgga

SEQ ID NO: 343 - RNA, 16 nt, Synthetic construct
  source 1..16: mol_type = other RNA
aaacaaaatgatggga

SEQ ID NO: 344 - RNA, 16 nt, Synthetic construct
  source 1..16: mol_type = other RNA
ttgtaggactaaataa

SEQ ID NO: 345 - RNA, 16 nt, Synthetic construct
  source 1..16: mol_type = other RNA
aggactaaataatgaa

SEQ ID NO: 346 - RNA, 16 nt, Synthetic construct
  source 1..16: mol_type = other RNA
actaaataatgatcta

SEQ ID NO: 347 - RNA, 16 nt, Synthetic construct
  source 1..16: mol_type = other RNA
taaataatgatcttca

SEQ ID NO: 348 - RNA, 16 nt, Synthetic construct
  source 1..16: mol_type = other RNA
atattagccaaagaga

SEQ ID NO: 349 - RNA, 16 nt, Synthetic construct
  source 1..16: mol_type = other RNA
ttagccaaagaggcaa

SEQ ID NO: 350 - RNA, 16 nt, Synthetic construct
  source 1..16: mol_type = other RNA
ccaaagaggcattcaa

SEQ ID NO: 351 - RNA, 16 nt, Synthetic construct
  source 1..16: mol_type = other RNA
caaagaggcattcaga

SEQ ID NO: 352 - RNA, 16 nt, Synthetic construct
  source 1..16: mol_type = other RNA
agaggcattcagcaaa

SEQ ID NO: 353 - RNA, 16 nt, Synthetic construct
  source 1..16: mol_type = other RNA
gcattcagcaattaaa

SEQ ID NO: 354 - RNA, 16 nt, Synthetic construct
  source 1..16: mol_type = other RNA
cattcagcaattaaaa

SEQ ID NO: 355 - RNA, 16 nt, Synthetic construct
  source 1..16: mol_type = other RNA
tcagcaattaaagaca

SEQ ID NO: 356 - RNA, 16 nt, Synthetic construct
  source 1..16: mol_type = other RNA
tcttttttgagtgtga

SEQ ID NO: 357 - RNA, 16 nt, Synthetic construct
  source 1..16: mol_type = other RNA
tttgagtgtgcaataa

SEQ ID NO: 358 - RNA, 16 nt, Synthetic construct
  source 1..16: mol_type = other RNA
ttgagtgtgcaatata

SEQ ID NO: 359 - RNA, 16 nt, Synthetic construct
  source 1..16: mol_type = other RNA
gcatttttcttggata

SEQ ID NO: 360 - RNA, 16 nt, Synthetic construct
  source 1..16: mol_type = other RNA
ttaacctttcaggaaa

SEQ ID NO: 361 - RNA, 16 nt, Synthetic construct
  source 1..16: mol_type = other RNA
ctttcaggaaagtata

SEQ ID NO: 362 - RNA, 16 nt, Synthetic construct
  source 1..16: mol_type = other RNA
tttcaggaaagtatca

SEQ ID NO: 363 - RNA, 16 nt, Synthetic construct
  source 1..16: mol_type = other RNA
tgataatagtaatgga

SEQ ID NO: 364 - RNA, 16 nt, Synthetic construct
  source 1..16: mol_type = other RNA
tagtaatggttctaga

SEQ ID NO: 365 - RNA, 16 nt, Synthetic construct
  source 1..16: mol_type = other RNA
ccttcaactgtataca

SEQ ID NO: 366 - RNA, 16 nt, Synthetic construct
  source 1..16: mol_type = other RNA
tgctccagtgttttca

SEQ ID NO: 367 - RNA, 16 nt, Synthetic construct
  source 1..16: mol_type = other RNA
attttcccacaattca

SEQ ID NO: 368 - RNA, 16 nt, Synthetic construct
  source 1..16: mol_type = other RNA
cacaattcttttgaaa

SEQ ID NO: 369 - RNA, 16 nt, Synthetic construct
  source 1..16: mol_type = other RNA
caattcttttgaaaga

SEQ ID NO: 370 - RNA, 16 nt, Synthetic construct
  source 1..16: mol_type = other RNA
gatggtaatcttttca

SEQ ID NO: 371 - RNA, 16 nt, Synthetic construct
  source 1..16: mol_type = other RNA
cgatgggatcattata

SEQ ID NO: 372 - RNA, 16 nt, Synthetic construct
  source 1..16: mol_type = other RNA
ggatcattatttcata

SEQ ID NO: 373 - RNA, 16 nt, Synthetic construct
  source 1..16: mol_type = other RNA
tcattatttcatgaca

SEQ ID NO: 374 - RNA, 16 nt, Synthetic construct
  source 1..16: mol_type = other RNA
ttgagaataaatgaga

SEQ ID NO: 375 - RNA, 16 nt, Synthetic construct
  source 1..16: mol_type = other RNA
gtaaggcctgctcaca

SEQ ID NO: 376 - RNA, 16 nt, Synthetic construct
  source 1..16: mol_type = other RNA
catgttcgctacctta

SEQ ID NO: 377 - RNA, 16 nt, Synthetic construct
  source 1..16: mol_type = other RNA
gttcgctaccttcaca

SEQ ID NO: 378 - RNA, 16 nt, Synthetic construct
  source 1..16: mol_type = other RNA
ttcgctaccttcacaa

SEQ ID NO: 379 - RNA, 16 nt, Synthetic construct
  source 1..16: mol_type = other RNA
gctaccttcacactta

SEQ ID NO: 380 - RNA, 16 nt, Synthetic construct
  source 1..16: mol_type = other RNA
taccttcacactttaa

SEQ ID NO: 381 - RNA, 16 nt, Synthetic construct
  source 1..16: mol_type = other RNA
ctgacatacgagctca

SEQ ID NO: 382 - RNA, 16 nt, Synthetic construct
  source 1..16: mol_type = other RNA
catgtgatttttgcta

SEQ ID NO: 383 - RNA, 16 nt, Synthetic construct
  source 1..16: mol_type = other RNA
atgtgatttttgctta

SEQ ID NO: 384 - RNA, 16 nt, Synthetic construct
  source 1..16: mol_type = other RNA
ttccctggattaagga

SEQ ID NO: 385 - RNA, 21 nt, Synthetic construct
  source 1..21: mol_type = other RNA
tcaaagtgagccttcttgttt

SEQ ID NO: 386 - RNA, 21 nt, Synthetic construct
  source 1..21: mol_type = other RNA
tacaaagtgagccttcttgtt

SEQ ID NO: 387 - RNA, 21 nt, Synthetic construct
  source 1..21: mol_type = other RNA
tcacaaagtgagccttcttgt

SEQ ID NO: 388 - RNA, 21 nt, Synthetic construct
  source 1..21: mol_type = other RNA
tgcacaaagtgagccttcttg

SEQ ID NO: 389 - RNA, 21 nt, Synthetic construct
  source 1..21: mol_type = other RNA
tgagcacaaagtgagccttct

SEQ ID NO: 390 - RNA, 21 nt, Synthetic construct
  source 1..21: mol_type = other RNA
tgcaatgttgagcacaaagtg

SEQ ID NO: 391 - RNA, 21 nt, Synthetic construct
  source 1..21: mol_type = other RNA
tttattcaggcaatgttgagc

SEQ ID NO: 392 - RNA, 21 nt, Synthetic construct
  source 1..21: mol_type = other RNA
tattctccttgcaataagtta

SEQ ID NO: 393 - RNA, 21 nt, Synthetic construct
  source 1..21: mol_type = other RNA
tataattccacagggctaaaa

SEQ ID NO: 394 - RNA, 21 nt, Synthetic construct
  source 1..21: mol_type = other RNA
tgataattccacagggctaaa

SEQ ID NO: 395 - RNA, 21 nt, Synthetic construct
  source 1..21: mol_type = other RNA
taggataattccacagggcta

SEQ ID NO: 396 - RNA, 21 nt, Synthetic construct
  source 1..21: mol_type = other RNA
tgcaattgaggataattccac

SEQ ID NO: 397 - RNA, 21 nt, Synthetic construct
  source 1..21: mol_type = other RNA
tgtgcaattgaggataattcc

SEQ ID NO: 398 - RNA, 21 nt, Synthetic construct
  source 1..21: mol_type = other RNA
tctcatcctctcctcctcatc

SEQ ID NO: 399 - RNA, 21 nt, Synthetic construct
  source 1..21: mol_type = other RNA
tgtaactcctccttctgccat

SEQ ID NO: 400 - RNA, 21 nt, Synthetic construct
  source 1..21: mol_type = other RNA
tctagtaactcctccttctgc

SEQ ID NO: 401 - RNA, 21 nt, Synthetic construct
  source 1..21: mol_type = other RNA
ttcactagtaactcctccttc

SEQ ID NO: 402 - RNA, 21 nt, Synthetic construct
  source 1..21: mol_type = other RNA
tataatcttcactagtaactc

SEQ ID NO: 403 - RNA, 21 nt, Synthetic construct
  source 1..21: mol_type = other RNA
tgataatcttcactagtaact

SEQ ID NO: 404 - RNA, 21 nt, Synthetic construct
  source 1..21: mol_type = other RNA
tgcgataatcttcactagtaa

SEQ ID NO: 405 - RNA, 21 nt, Synthetic construct
  source 1..21: mol_type = other RNA
tgctgtaaaaacgtgcgataa

SEQ ID NO: 406 - RNA, 21 nt, Synthetic construct
  source 1..21: mol_type = other RNA
tagaaggctgctgtaaaaacg

SEQ ID NO: 407 - RNA, 21 nt, Synthetic construct
  source 1..21: mol_type = other RNA
tcagaaggctgctgtaaaaac

SEQ ID NO: 408 - RNA, 21 nt, Synthetic construct
  source 1..21: mol_type = other RNA
tccagaaggctgctgtaaaaa

SEQ ID NO: 409 - RNA, 21 nt, Synthetic construct
  source 1..21: mol_type = other RNA
ttatttccagaaggctgctgt

SEQ ID NO: 410 - RNA, 21 nt, Synthetic construct
  source 1..21: mol_type = other RNA
tccatatttccagaaggctgc

SEQ ID NO: 411 - RNA, 21 nt, Synthetic construct
  source 1..21: mol_type = other RNA
tatccatatttccagaaggct

SEQ ID NO: 412 - RNA, 21 nt, Synthetic construct
  source 1..21: mol_type = other RNA
ttgtcatccatatttccagaa

SEQ ID NO: 413 - RNA, 21 nt, Synthetic construct
  source 1..21: mol_type = other RNA
tcactgtcatccatatttcca

SEQ ID NO: 414 - RNA, 21 nt, Synthetic construct
  source 1..21: mol_type = other RNA
taaaaaccactgtcatccata

SEQ ID NO: 415 - RNA, 21 nt, Synthetic construct
  source 1..21: mol_type = other RNA
ttagagaaaaaaccactgtca

SEQ ID NO: 416 - RNA, 21 nt, Synthetic construct
  source 1..21: mol_type = other RNA
tgaatagagaaaaaaccactg

SEQ ID NO: 417 - RNA, 21 nt, Synthetic construct
  source 1..21: mol_type = other RNA
ttgaatagagaaaaaaccact

SEQ ID NO: 418 - RNA, 21 nt, Synthetic construct
  source 1..21: mol_type = other RNA
taacctgaatagagaaaaaac

SEQ ID NO: 419 - RNA, 21 nt, Synthetic construct
  source 1..21: mol_type = other RNA
tgcattgcttataacctgaat

SEQ ID NO: 420 - RNA, 21 nt, Synthetic construct
  source 1..21: mol_type = other RNA
tggcattgcttataacctgaa

SEQ ID NO: 421 - RNA, 21 nt, Synthetic construct
  source 1..21: mol_type = other RNA
taggcattgcttataacctga

SEQ ID NO: 422 - RNA, 21 nt, Synthetic construct
  source 1..21: mol_type = other RNA
taaggcattgcttataacctg

SEQ ID NO: 423 - RNA, 21 nt, Synthetic construct
  source 1..21: mol_type = other RNA
tcaaggcattgcttataacct

SEQ ID NO: 424 - RNA, 21 nt, Synthetic construct
  source 1..21: mol_type = other RNA
ttttcaaggcattgcttataa

SEQ ID NO: 425 - RNA, 21 nt, Synthetic construct
  source 1..21: mol_type = other RNA
tctttcaaggcattgcttata

SEQ ID NO: 426 - RNA, 21 nt, Synthetic construct
  source 1..21: mol_type = other RNA
ttgaacaggattagttctaaa

SEQ ID NO: 427 - RNA, 21 nt, Synthetic construct
  source 1..21: mol_type = other RNA
tttgaacaggattagttctaa

SEQ ID NO: 428 - RNA, 21 nt, Synthetic construct
  source 1..21: mol_type = other RNA
tgttgaacaggattagttcta

SEQ ID NO: 429 - RNA, 21 nt, Synthetic construct
  source 1..21: mol_type = other RNA
tgactgttgaacaggattagt

SEQ ID NO: 430 - RNA, 21 nt, Synthetic construct
  source 1..21: mol_type = other RNA
tggactgttgaacaggattag

SEQ ID NO: 431 - RNA, 21 nt, Synthetic construct
  source 1..21: mol_type = other RNA
tactctggactgttgaacagg

SEQ ID NO: 432 - RNA, 21 nt, Synthetic construct
  source 1..21: mol_type = other RNA
tctgagcctctgatactctgg

SEQ ID NO: 433 - RNA, 21 nt, Synthetic construct
  source 1..21: mol_type = other RNA
tgtgttccttataattgcata

SEQ ID NO: 434 - RNA, 21 nt, Synthetic construct
  source 1..21: mol_type = other RNA
tagtgttccttataattgcat

SEQ ID NO: 435 - RNA, 21 nt, Synthetic construct
  source 1..21: mol_type = other RNA
tcagtgttccttataattgca

SEQ ID NO: 436 - RNA, 21 nt, Synthetic construct
  source 1..21: mol_type = other RNA
taccagtgttccttataattg

SEQ ID NO: 437 - RNA, 21 nt, Synthetic construct
  source 1..21: mol_type = other RNA
taaccagtgttccttataatt

SEQ ID NO: 438 - RNA, 21 nt, Synthetic construct
  source 1..21: mol_type = other RNA
ttaaaccagtgttccttataa

SEQ ID NO: 439 - RNA, 21 nt, Synthetic construct
  source 1..21: mol_type = other RNA
tgtaaaccagtgttccttata

SEQ ID NO: 440 - RNA, 21 nt, Synthetic construct
  source 1..21: mol_type = other RNA
ttgtaaaccagtgttccttat

SEQ ID NO: 441 - RNA, 21 nt, Synthetic construct
  source 1..21: mol_type = other RNA
tctgtaaaccagtgttcctta

SEQ ID NO: 442 - RNA, 21 nt, Synthetic construct
  source 1..21: mol_type = other RNA
taactgtaaaccagtgttcct

SEQ ID NO: 443 - RNA, 21 nt, Synthetic construct
  source 1..21: mol_type = other RNA
ttaactgtaaaccagtgttcc

SEQ ID NO: 444 - RNA, 21 nt, Synthetic construct
  source 1..21: mol_type = other RNA
tctaactgtaaaccagtgttc

SEQ ID NO: 445 - RNA, 21 nt, Synthetic construct
  source 1..21: mol_type = other RNA
tttctaactgtaaaccagtgt

SEQ ID NO: 446 - RNA, 21 nt, Synthetic construct
  source 1..21: mol_type = other RNA
tcactgttttcctaattttct

SEQ ID NO: 447 - RNA, 21 nt, Synthetic construct
  source 1..21: mol_type = other RNA
tccactgttttcctaattttc

SEQ ID NO: 448 - RNA, 21 nt, Synthetic construct
  source 1..21: mol_type = other RNA
taccactgttttcctaatttt

SEQ ID NO: 449 - RNA, 21 nt, Synthetic construct
  source 1..21: mol_type = other RNA
tagttaaaccactgttttcct

SEQ ID NO: 450 - RNA, 21 nt, Synthetic construct
  source 1..21: mol_type = other RNA
taagttaaaccactgttttcc

SEQ ID NO: 451 - RNA, 21 nt, Synthetic construct
  source 1..21: mol_type = other RNA
tcaagttaaaccactgttttc

SEQ ID NO: 452 - RNA, 21 nt, Synthetic construct
  source 1..21: mol_type = other RNA
tttcaagttaaaccactgttt

SEQ ID NO: 453 - RNA, 21 nt, Synthetic construct
  source 1..21: mol_type = other RNA
tattcaagttaaaccactgtt

SEQ ID NO: 454 - RNA, 21 nt, Synthetic construct
  source 1..21: mol_type = other RNA
ttctggacccgtcaagagaga

SEQ ID NO: 455 - RNA, 21 nt, Synthetic construct
  source 1..21: mol_type = other RNA
tgcaagatatgtatctgatat

SEQ ID NO: 456 - RNA, 21 nt, Synthetic construct
  source 1..21: mol_type = other RNA
tgtgcaagatatgtatctgat

SEQ ID NO: 457 - RNA, 21 nt, Synthetic construct
  source 1..21: mol_type = other RNA
tgtaattgagccaagaaaagt

SEQ ID NO: 458 - RNA, 21 nt, Synthetic construct
  source 1..21: mol_type = other RNA
tctgttgtaattgagccaaga

SEQ ID NO: 459 - RNA, 21 nt, Synthetic construct
  source 1..21: mol_type = other RNA
tcctgttgtaattgagccaag

SEQ ID NO: 460 - RNA, 21 nt, Synthetic construct
  source 1..21: mol_type = other RNA
ttcctgttgtaattgagccaa

SEQ ID NO: 461 - RNA, 21 nt, Synthetic construct
  source 1..21: mol_type = other RNA
tttcctgttgtaattgagcca

SEQ ID NO: 462 - RNA, 21 nt, Synthetic construct
  source 1..21: mol_type = other RNA
tcttcctgttgtaattgagcc

SEQ ID NO: 463 - RNA, 21 nt, Synthetic construct
  source 1..21: mol_type = other RNA
ttaaccttcctgttgtaattg

SEQ ID NO: 464 - RNA, 21 nt, Synthetic construct
  source 1..21: mol_type = other RNA
tataaccttcctgttgtaatt

SEQ ID NO: 465 - RNA, 21 nt, Synthetic construct
  source 1..21: mol_type = other RNA
tatagaataaccttcctgttg

SEQ ID NO: 466 - RNA, 21 nt, Synthetic construct
  source 1..21: mol_type = other RNA
tatatagaataaccttcctgt

SEQ ID NO: 467 - RNA, 21 nt, Synthetic construct
  source 1..21: mol_type = other RNA
taatatagaataaccttcctg

SEQ ID NO: 468 - RNA, 21 nt, Synthetic construct
  source 1..21: mol_type = other RNA
tcagcttcgcaatctggcaga

SEQ ID NO: 469 - RNA, 21 nt, Synthetic construct
  source 1..21: mol_type = other RNA
tataagttttggtcgatgcat

SEQ ID NO: 470 - RNA, 21 nt, Synthetic construct
  source 1..21: mol_type = other RNA
tcaataagttttggtcgatgc

SEQ ID NO: 471 - RNA, 21 nt, Synthetic construct
  source 1..21: mol_type = other RNA
tctccaataagttttggtcga

SEQ ID NO: 472 - RNA, 21 nt, Synthetic construct
  source 1..21: mol_type = other RNA
ttcttctccaataagttttgg

SEQ ID NO: 473 - RNA, 21 nt, Synthetic construct
  source 1..21: mol_type = other RNA
tctaattcttctccaataagt

SEQ ID NO: 474 - RNA, 21 nt, Synthetic construct
  source 1..21: mol_type = other RNA
tgtgctaattcttctccaata

SEQ ID NO: 475 - RNA, 21 nt, Synthetic construct
  source 1..21: mol_type = other RNA
tttatggactctttgctcttt

SEQ ID NO: 476 - RNA, 21 nt, Synthetic construct
  source 1..21: mol_type = other RNA
taggtctgttttatggactct

SEQ ID NO: 477 - RNA, 21 nt, Synthetic construct
  source 1..21: mol_type = other RNA
tttccaggtctgttttatgga

SEQ ID NO: 478 - RNA, 21 nt, Synthetic construct
  source 1..21: mol_type = other RNA
ttgagccatcatttgcttcta

SEQ ID NO: 479 - RNA, 21 nt, Synthetic construct
  source 1..21: mol_type = other RNA
taacattcctgagccatcatt

SEQ ID NO: 480 - RNA, 21 nt, Synthetic construct
  source 1..21: mol_type = other RNA
tttagtgccagagccctctgc

SEQ ID NO: 481 - RNA, 21 nt, Synthetic construct
  source 1..21: mol_type = other RNA
ttcaatttcttggcgacttag

SEQ ID NO: 482 - RNA, 21 nt, Synthetic construct
  source 1..21: mol_type = other RNA
taccttgcatacttagctgaa

SEQ ID NO: 483 - RNA, 21 nt, Synthetic construct
  source 1..21: mol_type = other RNA
ttaccttgcatacttagctga

SEQ ID NO: 484 - RNA, 21 nt, Synthetic construct
  source 1..21: mol_type = other RNA
taactaccttgcatacttagc

SEQ ID NO: 485 - RNA, 21 nt, Synthetic construct
  source 1..21: mol_type = other RNA
tgaactaccttgcatacttag

SEQ ID NO: 486 - RNA, 21 nt, Synthetic construct
  source 1..21: mol_type = other RNA
tggaactaccttgcatactta

SEQ ID NO: 487 - RNA, 21 nt, Synthetic construct
  source 1..21: mol_type = other RNA
ttggaactaccttgcatactt

SEQ ID NO: 488 - RNA, 21 nt, Synthetic construct
  source 1..21: mol_type = other RNA
tctggaactaccttgcatact

SEQ ID NO: 489 - RNA, 21 nt, Synthetic construct
  source 1..21: mol_type = other RNA
ttctggaactaccttgcatac

SEQ ID NO: 490 - RNA, 21 nt, Synthetic construct
  source 1..21: mol_type = other RNA
ttttctggaactaccttgcat

SEQ ID NO: 491 - RNA, 21 nt, Synthetic construct
  source 1..21: mol_type = other RNA
tgtacctgatgtctgtgtcat

SEQ ID NO: 492 - RNA, 21 nt, Synthetic construct
  source 1..21: mol_type = other RNA
tttgtacctgatgtctgtgtc

SEQ ID NO: 493 - RNA, 21 nt, Synthetic construct
  source 1..21: mol_type = other RNA
tatttgtacctgatgtctgtg

SEQ ID NO: 494 - RNA, 21 nt, Synthetic construct
  source 1..21: mol_type = other RNA
tgaagtaagatttgtacctga

SEQ ID NO: 495 - RNA, 21 nt, Synthetic construct
  source 1..21: mol_type = other RNA
tgaagctcttctgaagtaaga

SEQ ID NO: 496 - RNA, 21 nt, Synthetic construct
  source 1..21: mol_type = other RNA
tccgaagctcttctgaagtaa

SEQ ID NO: 497 - RNA, 21 nt, Synthetic construct
  source 1..21: mol_type = other RNA
ttccgaagctcttctgaagta

SEQ ID NO: 498 - RNA, 21 nt, Synthetic construct
  source 1..21: mol_type = other RNA
tctcttccgaagctcttctga

SEQ ID NO: 499 - RNA, 21 nt, Synthetic construct
  source 1..21: mol_type = other RNA
tcttctcgtctcttccgaagc

SEQ ID NO: 500 - RNA, 21 nt, Synthetic construct
  source 1..21: mol_type = other RNA
tgcttctcgtctcttccgaag

SEQ ID NO: 501 - RNA, 21 nt, Synthetic construct
  source 1..21: mol_type = other RNA
tgtaggcttctcgtctcttcc

SEQ ID NO: 502 - RNA, 21 nt, Synthetic construct
  source 1..21: mol_type = other RNA
tagtaggcttctcgtctcttc

SEQ ID NO: 503 - RNA, 21 nt, Synthetic construct
  source 1..21: mol_type = other RNA
taagtaggcttctcgtctctt

SEQ ID NO: 504 - RNA, 21 nt, Synthetic construct
  source 1..21: mol_type = other RNA
taaagtaggcttctcgtctct

SEQ ID NO: 505 - RNA, 21 nt, Synthetic construct
  source 1..21: mol_type = other RNA
tcaaagtaggcttctcgtctc

SEQ ID NO: 506 - RNA, 21 nt, Synthetic construct
  source 1..21: mol_type = other RNA
ttcaaagtaggcttctcgtct

SEQ ID NO: 507 - RNA, 21 nt, Synthetic construct
  source 1..21: mol_type = other RNA
tgtttttcaaagtaggcttct

SEQ ID NO: 508 - RNA, 21 nt, Synthetic construct
  source 1..21: mol_type = other RNA
tcacctagatcactcccaagt

SEQ ID NO: 509 - RNA, 21 nt, Synthetic construct
  source 1..21: mol_type = other RNA
ttcacctagatcactcccaag

SEQ ID NO: 510 - RNA, 21 nt, Synthetic construct
  source 1..21: mol_type = other RNA
tatcacctagatcactcccaa

SEQ ID NO: 511 - RNA, 21 nt, Synthetic construct
  source 1..21: mol_type = other RNA
tcatagcatcacctagatcac

SEQ ID NO: 512 - RNA, 21 nt, Synthetic construct
  source 1..21: mol_type = other RNA
tgacatggtcacagctgcctg

SEQ ID NO: 513 - RNA, 21 nt, Synthetic construct
  source 1..21: mol_type = other RNA
taaagacatggtcacagctgc

SEQ ID NO: 514 - RNA, 21 nt, Synthetic construct
  source 1..21: mol_type = other RNA
ttaaagacatggtcacagctg

SEQ ID NO: 515 - RNA, 21 nt, Synthetic construct
  source 1..21: mol_type = other RNA
tctaaagacatggtcacagct

SEQ ID NO: 516 - RNA, 21 nt, Synthetic construct
  source 1..21: mol_type = other RNA
ttctaaagacatggtcacagc

SEQ ID NO: 517 - RNA, 21 nt, Synthetic construct
  source 1..21: mol_type = other RNA
tttctaaagacatggtcacag

SEQ ID NO: 518 - RNA, 21 nt, Synthetic construct
  source 1..21: mol_type = other RNA
tcagtttctaaagacatggtc

SEQ ID NO: 519 - RNA, 21 nt, Synthetic construct
  source 1..21: mol_type = other RNA
tgacagtttctaaagacatgg

SEQ ID NO: 520 - RNA, 21 nt, Synthetic construct
  source 1..21: mol_type = other RNA
ttctgacagtttctaaagaca

SEQ ID NO: 521 - RNA, 21 nt, Synthetic construct
  source 1..21: mol_type = other RNA
ttcaaatcatttctgacagtt

SEQ ID NO: 522 - RNA, 21 nt, Synthetic construct
  source 1..21: mol_type = other RNA
tatgctgtaatcacacaggat

SEQ ID NO: 523 - RNA, 21 nt, Synthetic construct
  source 1..21: mol_type = other RNA
ttatgctgtaatcacacagga

SEQ ID NO: 524 - RNA, 21 nt, Synthetic construct
  source 1..21: mol_type = other RNA
tctatgctgtaatcacacagg

SEQ ID NO: 525 - RNA, 21 nt, Synthetic construct
  source 1..21: mol_type = other RNA
taaagtggaccctatgctgta

SEQ ID NO: 526 - RNA, 21 nt, Synthetic construct
  source 1..21: mol_type = other RNA
tacattaccaaagtggaccct

SEQ ID NO: 527 - RNA, 21 nt, Synthetic construct
  source 1..21: mol_type = other RNA
tgacacattaccaaagtggac

SEQ ID NO: 528 - RNA, 21 nt, Synthetic construct
  source 1..21: mol_type = other RNA
ttgacacattaccaaagtgga

SEQ ID NO: 529 - RNA, 21 nt, Synthetic construct
  source 1..21: mol_type = other RNA
tttgacacattaccaaagtgg

SEQ ID NO: 530 - RNA, 21 nt, Synthetic construct
  source 1..21: mol_type = other RNA
tctctttgacacattaccaaa

SEQ ID NO: 531 - RNA, 21 nt, Synthetic construct
  source 1..21: mol_type = other RNA
ttcatctctttgacacattac

SEQ ID NO: 532 - RNA, 21 nt, Synthetic construct
  source 1..21: mol_type = other RNA
tctcatctctttgacacatta

SEQ ID NO: 533 - RNA, 21 nt, Synthetic construct
  source 1..21: mol_type = other RNA
tattttgtttgcaaaccgcta

SEQ ID NO: 534 - RNA, 21 nt, Synthetic construct
  source 1..21: mol_type = other RNA
tccatcattttgtttgcaaac

SEQ ID NO: 535 - RNA, 21 nt, Synthetic construct
  source 1..21: mol_type = other RNA
tcccatcattttgtttgcaaa

SEQ ID NO: 536 - RNA, 21 nt, Synthetic construct
  source 1..21: mol_type = other RNA
ttatttagtcctacaaccgac

SEQ ID NO: 537 - RNA, 21 nt, Synthetic construct
  source 1..21: mol_type = other RNA
ttcattatttagtcctacaac

SEQ ID NO: 538 - RNA, 21 nt, Synthetic construct
  source 1..21: mol_type = other RNA
tagatcattatttagtcctac

SEQ ID NO: 539 - RNA, 21 nt, Synthetic construct
  source 1..21: mol_type = other RNA
tgaagatcattatttagtcct

SEQ ID NO: 540 - RNA, 21 nt, Synthetic construct
  source 1..21: mol_type = other RNA
tctctttggctaatatttgga

SEQ ID NO: 541 - RNA, 21 nt, Synthetic construct
  source 1..21: mol_type = other RNA
ttgcctctttggctaatattt

SEQ ID NO: 542 - RNA, 21 nt, Synthetic construct
  source 1..21: mol_type = other RNA
ttgaatgcctctttggctaat

SEQ ID NO: 543 - RNA, 21 nt, Synthetic construct
  source 1..21: mol_type = other RNA
tctgaatgcctctttggctaa

SEQ ID NO: 544 - RNA, 21 nt, Synthetic construct
  source 1..21: mol_type = other RNA
tttgctgaatgcctctttggc

SEQ ID NO: 545 - RNA, 21 nt, Synthetic construct
  source 1..21: mol_type = other RNA
tttaattgctgaatgcctctt

SEQ ID NO: 546 - RNA, 21 nt, Synthetic construct
  source 1..21: mol_type = other RNA
ttttaattgctgaatgcctct

SEQ ID NO: 547 - RNA, 21 nt, Synthetic construct
  source 1..21: mol_type = other RNA
tgtctttaattgctgaatgcc

SEQ ID NO: 548 - RNA, 21 nt, Synthetic construct
  source 1..21: mol_type = other RNA
tcacactcaaaaaagaaaaag

SEQ ID NO: 549 - RNA, 21 nt, Synthetic construct
  source 1..21: mol_type = other RNA
ttattgcacactcaaaaaaga

SEQ ID NO: 550 - RNA, 21 nt, Synthetic construct
  source 1..21: mol_type = other RNA
tatattgcacactcaaaaaag

SEQ ID NO: 551 - RNA, 21 nt, Synthetic construct
  source 1..21: mol_type = other RNA
tatccaagaaaaatgccctaa

SEQ ID NO: 552 - RNA, 21 nt, Synthetic construct
  source 1..21: mol_type = other RNA
tttcctgaaaggttaatttgg

SEQ ID NO: 553 - RNA, 21 nt, Synthetic construct
  source 1..21: mol_type = other RNA
tatactttcctgaaaggttaa

SEQ ID NO: 554 - RNA, 21 nt, Synthetic construct
  source 1..21: mol_type = other RNA
tgatactttcctgaaaggtta

SEQ ID NO: 555 - RNA, 21 nt, Synthetic construct
  source 1..21: mol_type = other RNA
tccattactattatcaacatc

SEQ ID NO: 556 - RNA, 21 nt, Synthetic construct
  source 1..21: mol_type = other RNA
tctagaaccattactattatc

SEQ ID NO: 557 - RNA, 21 nt, Synthetic construct
  source 1..21: mol_type = other RNA
tgtatacagttgaagggagaa

SEQ ID NO: 558 - RNA, 21 nt, Synthetic construct
  source 1..21: mol_type = other RNA
tgaaaacactggagcacacgg

SEQ ID NO: 559 - RNA, 21 nt, Synthetic construct
  source 1..21: mol_type = other RNA
tgaattgtgggaaaataatga

SEQ ID NO: 560 - RNA, 21 nt, Synthetic construct
  source 1..21: mol_type = other RNA
tttcaaaagaattgtgggaaa

SEQ ID NO: 561 - RNA, 21 nt, Synthetic construct
  source 1..21: mol_type = other RNA
tctttcaaaagaattgtggga

SEQ ID NO: 562 - RNA, 21 nt, Synthetic construct
  source 1..21: mol_type = other RNA
tgaaaagattaccatctttca

SEQ ID NO: 563 - RNA, 21 nt, Synthetic construct
  source 1..21: mol_type = other RNA
tataatgatcccatcgtaggg

SEQ ID NO: 564 - RNA, 21 nt, Synthetic construct
  source 1..21: mol_type = other RNA
tatgaaataatgatcccatcg

SEQ ID NO: 565 - RNA, 21 nt, Synthetic construct
  source 1..21: mol_type = other RNA
tgtcatgaaataatgatccca

SEQ ID NO: 566 - RNA, 21 nt, Synthetic construct
  source 1..21: mol_type = other RNA
tctcatttattctcaagtact

SEQ ID NO: 567 - RNA, 21 nt, Synthetic construct
  source 1..21: mol_type = other RNA
tgtgagcaggccttacctaga

SEQ ID NO: 568 - RNA, 21 nt, Synthetic construct
  source 1..21: mol_type = other RNA
taaggtagcgaacatgatgaa

SEQ ID NO: 569 - RNA, 21 nt, Synthetic construct
  source 1..21: mol_type = other RNA
tgtgaaggtagcgaacatgat

SEQ ID NO: 570 - RNA, 21 nt, Synthetic construct
  source 1..21: mol_type = other RNA
ttgtgaaggtagcgaacatga

SEQ ID NO: 571 - RNA, 21 nt, Synthetic construct
  source 1..21: mol_type = other RNA
taagtgtgaaggtagcgaaca

SEQ ID NO: 572 - RNA, 21 nt, Synthetic construct
  source 1..21: mol_type = other RNA
ttaaagtgtgaaggtagcgaa

SEQ ID NO: 573 - RNA, 21 nt, Synthetic construct
  source 1..21: mol_type = other RNA
tgagctcgtatgtcagataaa

SEQ ID NO: 574 - RNA, 21 nt, Synthetic construct
  source 1..21: mol_type = other RNA
tagcaaaaatcacatggagct

SEQ ID NO: 575 - RNA, 21 nt, Synthetic construct
  source 1..21: mol_type = other RNA
taagcaaaaatcacatggagc

SEQ ID NO: 576 - RNA, 21 nt, Synthetic construct
  source 1..21: mol_type = other RNA
tccttaatccagggaaattta